(19) United States
(12) Patent Application Publication
(10) Pub. No.: US 2012/0122923 A1
(43) Pub. Date: May 17, 2012

(54) DUAL MOLECULES CONTAINING A PEROXIDE DERIVATIVE, THEIR SYNTHESIS AND THERAPEUTIC USES

(75) Inventors: Frederic COSLEDAN, Labege (FR); Bernard MEUNIER, Castanet (FR); Alain PELLET, Antony (FR)

(73) Assignees: SANOFI-AVENTIS, Paris (FR); PALUMED S.A., Castanet Tolosan (FR); CENTRE NATIONAL DE LA RECHERCHE SCIENTIFIQUE (C.N.R.S.), Paris (FR)

(21) Appl. No.: 12/332,633

(22) Filed: Dec. 11, 2008

Related U.S. Application Data
(63) Continuation of application No. PCT/FR2007/000946, filed on Jun. 8, 2007.

(30) Foreign Application Priority Data
Jun. 13, 2006 (FR) 0605235

(51) Int. Cl. A61K 31/47; A61P 33/06; C07D 405/12

(52) U.S. Cl. 514/313; 546/160; 546/171; 514/314

(57) ABSTRACT

The invention concerns dual molecules corresponding to formula (I): in which: A represents a molecular residue with antimalarial activity of formula (IIa) or (IIIa) or a residue facilitating bioavailability; B represents a cycloalkyl group potentially substituted, or B represents a bi- or tricyclic group capable of being substituted, or B represents 2 cycloalkyl groups linked together through either a single bond or an alkylene chain; m and n represent independently of one another 0, 1 or 2; R₅ represents a hydrogen atom, an alkyl, cycloalkyl or C₁₋₃-alkylene-cycloakyl group; Z₁ and Z₂ represent an alkyl radical, the group Z₁+Z₂+Cᵢ+Cⱼ representing a mono- or polycyclic structure, with one of the Z₁ or Z₂ being able to represent a single bond; R₁ and R₂, identical or different, represent a hydrogen atom or a functional group capable of increasing hydrosolubility; Rₓ and R_{y} forming together a cyclic peroxide including 4 to 8 links and including 1 or 2 additional oxygen atoms in the cyclic structure, possibly substituted by one or more R₃ groups; as a base or a salt to be added to an acid, as a hydrate or solvate, in racemic form, isomers and their mixtures, in addition to their diasteroisomers and their mixtures. Preparation method and use as medications with antimalarial activity.

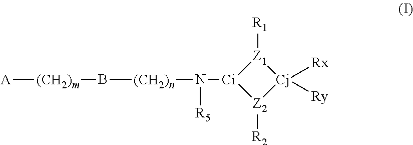

The invention relates to hybrid molecules containing a peroxide derivative, in particular having an antimalarial activity, to the synthesis thereof and to therapeutic applications thereof.

Malaria is one of the primary infectious causes of mortality in the world, affecting 100 to 200 million people every year. The significant upsurge in the disease observed in recent years is due to a number of factors, including:
vectors, namely Anopheles, which are becoming resistant to conventional and cheap insecticides such as DDT (abbreviation for 1,1,1-trichloro-2,2-bis(p-chlorophenyl)-ethane);
population growth in zones at risk; and, principally,
the resistance of many strains of Plasmodium falciparum, the parasite responsible for the deadly forms of the disease, to conventional medicinal products such as chloroquine and mefloquine.

The discovery of artemisinin, a powerful antimalarial extracted from Artemisia annua, has drawn attention to molecules having, like artemisinin, an endoperoxide function. Artemisinin and certain of its hemi-synthetic derivatives such as artemether and artesunate have proved to be very active on resistant strains of P. falciparum. However, the high cost of those compounds of natural origin and uncertain supply limit their use. Thus, there is an interest in synthetic antimalarial compounds which are cheap and accessible. Furthermore, such molecules are generally strongly metabolized, rendering their use as a therapeutic substance more difficult.

International applications published with numbers WO 01/77105 and WO2005/04619 describe hybrid molecules constituted by a compound endowed with antimalarial properties and a peroxide type derivative. However, those coupled products, while effective, are strongly metabolized.

Thus, it appears to be necessary to investigate novel compounds with an effective antimalarial activity while having improved pharmacological properties, especially ADME (absorption, distribution, metabolism, elimination properties), rendering them particularly suitable for use as a medicinal product.

To this end, the inventors have developed a novel family of hybrid molecules having an effective antimalarial activity and which also have improved ADME properties. This novel family of molecules, corresponding to compounds with formula (I) described below, has improved metabolic stability on human hepatic microsomes, thus confirming the importance of the compounds of the invention for use as a medicinal product.

Thus, the invention pertains to compounds with formula (I), to the synthesis thereof and to their biological applications, especially for the treatment of parasitic diseases such as malaria.

The invention concerns compounds with formula (I):

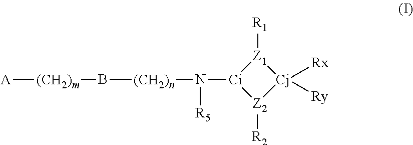

in which:

A represents:
a residue of a molecule with antimalarial activity selected from:
   an aminoquinoline with formula (IIa):

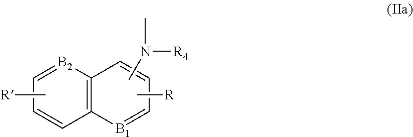

in which:
R and R′, which may be identical or different, each represent one or more (for example 1 to 5) substituents occupying distinct positions on the cycles to which they are attached, selected from:
   a hydrogen or halogen atom, a —OH, —CF₃, —OCF₃, aryl, —O-aryl, heteroaryl, alkyl or —O-alkyl group, said alkyl groups containing 1 to 5 carbon atoms;
   a cycloalkyl or —O-cycloalkyl group, said cycloalkyl groups possibly containing 3 to 5 carbon atoms;
   —NO₂ or —N(Rₐ,R_{b}), where Rₐ and R_{b}, which may be identical or different, each independently represent a hydrogen atom or an alkyl group containing 1 to 5 carbon atoms;

or Rₐ and R_{b}, which may be identical or different, represent a cycloalkyl group which may contain 3 to 5 carbon atoms;

or Rₐ and R_{b} together with the nitrogen atom to which they are attached form a pyrrolidinyl or piperidinyl group;
R₄ represents a hydrogen atom or an alkyl group which may contain 1 to 5 carbon atoms or R₄ represents a cycloalkyl group which may contain 3 to carbon atoms;
B₁ represents a nitrogen atom and B₂ represents a —CH=link, or B₁ represents a —CH=link and B₂ represents a nitrogen atom;
a group with formula (IIIa):

R₆—CHOH— (IIIa)

in which R₆ represents an aryl radical, preferably a 9-phenanthrenyl or a nitrogenous heterocyclic residue, preferably a 4-quinolinyl optionally substituted with one or more (for example 1 to 5) groups R as defined for the compound with formula (IIa);
or A represents a residue facilitating bioavailability, said residue having one or more heteroatoms selected from N, O and S in a mono- or poly-cyclic molecule which may contain 6 to 18 carbon atoms, which may be saturated or unsaturated or in a chain which may contain 1 to 18 linear carbon atoms, optionally substituted, such as a guanidinium, morpholino, peptide or polyamine residue;

B represents a cycloalkyl group which may contain 3 to 8 carbon atoms, optionally substituted with one or more groups selected from: a halogen atom, a hydroxyl group, an alkyl group which may contain 1 to 6 carbon atoms or a cycloalkyl group which may contain 3 to 6 carbon atoms;
or B represents a bi- or tri-cyclic group which may contain 4 to 18 carbon atoms, optionally substituted with one or more groups selected from a halogen atom, a hydroxyl group, an alkyl group which may contain 1 to 6 carbon atoms or a cycloalkyl group which may contain 3 to 6 carbon atoms;
or B represents 2 cycloalkyl groups which may contain 3 to 6 carbon atoms, said cycloalkyls being connected together via a single bond or an alkylene chain which may contain 1 or 2 carbon atoms;

m and n independently represent 0, 1 or 2;

R₅ represents a hydrogen atom or an alkyl group, a —C(O)-alkyl group or a —C(O)O-alkyl group, said alkyl groups possibly containing 1 to 5 carbon atoms;
or R₅ represents a cycloalkyl group, a —C(O)-cycloalkyl group, a —C(O)O-cycloalkyl group or a C₁₋₃-alkylene-cycloalkyl group, said cycloalkyl groups possibly containing 3 to 6 carbon atoms;

Z₁ and Z₂, which may be identical or different, represent an alkylene radical which may contain 1 to 4 saturated or unsaturated carbon atoms, the entity Z₁+Z₂+Ci+Cj thus representing:
either a cycloalkyl group which may contain 3 to 10 carbon atoms;
or a poly-cyclic structure which may contain 4 to 18 carbon atoms; Z₁ or Z₂ possibly representing a single bond between the carbon atoms Ci and Cj, it being understood that Z₁ and Z₂ cannot both represent a single bond at the same time;

R₁ and R₂, which may be identical or different, represent a hydrogen atom or a functional group which is capable of enhancing hydrosolubility;

Rₓ and R_{y} together form a cyclic peroxide containing 4 to 8 links and containing 1 or 2 supplemental oxygen atoms in the cyclic structure (i.e. a total of 3 or 4 oxygen atoms in the cycle), Cj being one of the vertices of said cyclic peroxide;

said cyclic peroxide being substituted with a group R₃, R₃ representing 1 to 8 groups which may be identical or different, occupying any positions on the carbon atoms of the peroxide cycle and being selected from the following atoms and groups:
hydrogen, halogen, a —OH, —CF₃, —NO₂, —OCF₃, aryl, —O-aryl, heteroaryl, alkyl or —O-alkyl group, said alkyl groups containing 1 to 10 carbon atoms;
a cycloalkyl group possibly containing 3 to 7 carbon atoms and possibly further containing 1 to 3 heteroatoms selected from oxygen, nitrogen and sulphur, optionally substituted with one or more groups (for example 1 to 8) selected from a halogen atom, a hydroxyl group, an alkyl group which may contain 1 to 8 carbon atoms or a cycloalkyl group which may contain 3 to 8 carbon atoms;
an —O-cycloalkyl group which may contain 3 to 7 carbon atoms;
a bi- or tri-cyclic group which may contain 4 to 18 carbon atoms and may also contain 1 to 6 heteroatoms selected from oxygen, nitrogen and sulphur, optionally substituted with one or more groups selected from a halogen atom, a hydroxyl group, an alkyl group which may contain 1 to 8 carbon atoms or a cycloalkyl group which may contain 3 to 8 carbon atoms;
or two groups R₃ carried by adjacent carbon atoms on the peroxide cycle may together form a saturated or unsaturated cycloalkyl group containing 5 or 6 carbon atoms, said group R₃ itself possibly being substituted with 1 to 6 substituents R₃ as defined above;
or two groups R₃ carried by the same carbon atom of the peroxide cycle may together form a cycloalkyl group which may contain 3 to 7 carbon atoms or a bi- or tri-cyclic group which may contain 4 to 18 carbon atoms (which will thus be located in the Spiro position on the peroxide cycle).

Advantageously, residue A drains the compound with formula (I) in accordance with the invention into the interior of the parasite, which then exerts an alkylating effect on heme and/or the parasitic proteins.

The compounds with formula (I) may exist as bases or addition salts with acids. Such addition salts also form part of the invention. Said salts are advantageously prepared with pharmaceutically acceptable acids, but the salts of other useful acids, for purification or isolation of compounds with formula (I), also form part of the invention.

The compounds of the invention may also exist in the form of hydrates or solvates, namely in the form of associations or combinations with one or more molecules of water or with a solvent. Such hydrates and solvates also form part of the invention.

The invention encompasses mixtures of diastereoisomers in all proportions, as well as pure diastereoisomers with formula (I). The invention also encompasses racemic mixtures, as well as optically pure isomers of molecules with formula (I), again in mixtures in all proportions of said optically pure isomers. The invention also encompasses achiral molecules.

In the definition of compounds with formula (I) above and below, the following meanings are intended, unless otherwise mentioned in the text:

halogen atom: a fluorine, chlorine, bromine or iodine atom;

alkyl group: a saturated, linear or branched monovalent aliphatic group.

Examples which may be cited are methyl, ethyl, propyl, isopropyl, butyl, isobutyl, tert-butyl and pentyl groups;

alkylene radical or chain: a saturated, linear or branched divalent aliphatic group. Examples of a C₁₋₃ alkylene group which represents a linear or branched divalent carbon chain containing 1 to 3 carbon atoms are methylenyl (—CH₂—), ethylenyl (—CH₂CH₂—), 1-methylethylenyl (—CH(CH₃)CH₂—) and propylenyl (—CH₂CH₂CH₂—);

cycloalkyl group: a saturated cyclic aliphatic group. Examples which may be cited are cyclopropyl, methylcyclopropyl, cyclobutyl, cyclopentyl and cyclohexyl groups;

a bi-cyclic structure: a structure comprising 2 saturated cyclic aliphatic groups containing 4 to 18 carbon atoms, said groups possibly being:

fused, i.e. they have a bond in common.

An example which may be cited is the perhydronaphthyl group:

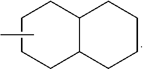

or bridged, i.e. at least 2 atoms of the bi-cyclic structure are connected via a single bond or a carbonaceous chain which may contain 1 to 4 carbon atoms.
An example which may be cited is:

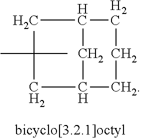

or a spiro junction, i.e. they are connected via a common carbon atom.
An example which may be cited is the cyclopentane-spiro-cyclobutyl group:

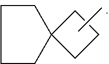

a tri-cyclic structure: a structure comprising 3 saturated cyclic aliphatic groups containing 4 to 18 carbon atoms, said groups possibly being fused (as defined above) or bridged (as defined above).

An example of a fused tri-cyclic structure which may be cited is the perhydroanthracene group:

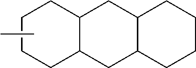

An example of a bridged tri-cyclic structure which may be cited is the adamantyl group which is a tri-cyclic structure containing 10 carbon atoms:

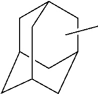

a poly-cyclic structure: a bi- or tri-cyclic structure as defined above;

a cyclic peroxide group: a cyclic alkyl group containing 2 adjacent oxygen atoms;

an aryl group: a mono-cyclic or poly-cyclic aromatic system containing 6 to 18 carbon atoms, preferably 6 to 14 carbon atoms and more preferably 6 to 10 carbon atoms. When the system is poly-cyclic, at least one of the cycles is aromatic. Examples which may be cited are the phenyl, naphthyl, tetrahydronaphthyl and indanyl groups;

a heteroaryl group: a mono-cyclic or poly-cyclic aromatic system comprising 5 to 18 links, preferably 5 to 14 links and more preferably 5 to 10 links and comprising one or more heteroatoms such as nitrogen, oxygen or sulphur atoms. When the system is poly-cyclic, at least one of the cycles is aromatic. The nitrogen atoms may be in the form of N-oxides. Examples of mono-cyclic heteroaryl groups which may be cited are thiazolyl, thiadiazolyl, thienyl, imidazolyl, triazolyl, tetrazolyl, pyridinyl, furanyl, oxazolyl, isoxazolyl, oxadiazolyl, pyrrolyl, pyrazolyl, pyrimidinyl and pyridazinyl groups. Examples of bi-cyclic heteroaryl groups which may be cited are indolyl, benzofuranyl, chromen-2-on-yl, benzimidazolyl, benzothienyl, benzotriazolyl, benzothiazolyl, benzoxazolyl, quinolinyl, isoquinolinyl, indazolyl, indolizinyl, quinazolinyl, phthalazinyl, quinoxalinyl, naphthyridinyl, 2,3-dihydro-1H-indolyl, 2,3-dihydro-benzofuranyl, tetrahydroquinolinyl and tetrahydroisoquinolinyl;

residue facilitating bioavailability:
a saturated or unsaturated cycloalkyl group which may contain 6 to 8 carbon atoms, said cycloalkyl group comprising one or more heteroatoms selected from N, O and S;
a saturated or unsaturated bi- or tri-cyclic group which may contain 6 to 18 carbon atoms, said bi- or tri-cyclic groups comprising one or more heteroatoms selected from N, O and S;
a linear, optionally substituted carbonaceous chain which may contain 1 to 18 carbon atoms, said chain comprising one or more heteroatoms selected from N, O and S.
   Examples of residues facilitating bioavailability which may be cited are guanidinium, morpholino, peptide or polyamine residues;

functional group capable of enhancing the hydrosolubility of the dual molecule: a group advantageously selected from —COOH, —OH or —N(Rₐ,R_{b}) where Rₐ and R_{b}, which may be identical or different, represent a hydrogen atom, an alkyl group which may contain 1 to 5 carbon atoms or a cycloalkyl group which may contain 3 to 5 carbon atoms.

Compounds in accordance with the invention which may be cited include a first group of compounds with formula (I) in which A, B, m, n, Z₁, Z₂, the entity Z₁+Z₂+Ci+Cj, R₁, R₂, Rₓ, R_{y} are as defined above and R₅ represents a hydrogen atom or an alkyl group, a —C(O)-alkyl group or a —C(O)O-alkyl group, said alkyl groups possibly containing 1 to 5 carbon atoms;
or R₅ represents a cycloalkyl group, a —C(O)-cycloalkyl group or a —C(O)O-cycloalkyl group, said cycloalkyl groups possibly containing 3 to 6 carbon atoms.
Compounds in accordance with the invention which may be cited include a second group of compounds with formula (I) in which:

A represents an aminoquinoline with formula (IIa):

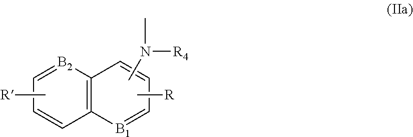

in which:

R and R′, which may be identical or different, each represent one or more (for example 1 to 5) substituents occupying distinct positions on the cycles to which they are attached, selected from:
a hydrogen or halogen atom, a —OH, —CF₃, —OCF₃, aryl, —O-aryl, heteroaryl, alkyl or —O-alkyl group, said alkyl groups containing 1 to 5 carbon atoms;
a cycloalkyl group or —O-cycloalkyl group, said cycloalkyl groups possibly containing 3 to 5 carbon atoms;
—NO₂ or —N(Rₐ,R_{b}), where Rₐ and R_{b}, which may be identical or different, represent hydrogen atoms or an alkyl group containing 1 to 5 carbon atoms;

or Rₐ and R_{b}, which may be identical or different, represent a cycloalkyl group which may contain 3 to 5 carbon atoms;

or Rₐ and R_{b} together with the nitrogen atom to which they are attached form a pyrrolidinyl or piperidinyl group;
R₄ represents a hydrogen atom, an alkyl group which may contain 1 to 5 carbon atoms or R₄ represents a cycloalkyl group which may contain 3 to 5 carbon atoms;
B₁ represents a nitrogen atom and B₂ represents a —CH=link;

or B₁ represents a —CH=link and B₂ represents a nitrogen atom.

Compounds in accordance with the invention which may be cited include a third group of compounds with formula (I) in which A represents an aminoquinoline with formulae (IIb) or (IIc) below:

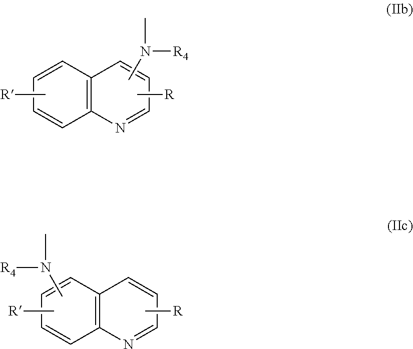

in which R, R′ and R₄ are as defined for the compound with formula (IIa).

Compounds in accordance with the invention which may be cited include a fourth group of compounds with formula (I) in which B represents a group selected from:

cis-1,2-methylenecyclopentyl, trans-1,2-cyclohexyl, cis-1,2-cyclohexyl, cis-1,2-methylenecyclohexyl, trans-1,4-cyclohexyl, cis-1,4-cyclohexyl, a cis/trans-1,4-cyclohexyl mixture, a cis/trans-1,3-cyclohexyl mixture, a cis/trans-1,3-dimethylenecyclohexyl mixture, cis-1,4-dimethylenecyclohexyl and 4,4′-methylene-bis-cyclohexane.

Compounds in accordance with the invention which may be cited include a fifth group of compounds with formula (I) in which A represents a nitrogenous heterocycle of the aminoquinoline type with formula (IIa) and which satisfies formula (I.1) below:

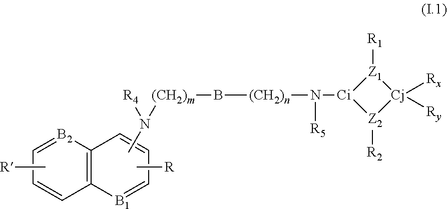

in which R, R′, B₁, B₂ and R₄ are as defined for the compound with formula (IIa) and B, Z₁, Z₂, Ci, Cj, R₁, R₂, Rₓ, R_{y}, R₅, m and n are as defined for the compound with formula (I).

In compounds with formula (I), Rₓ and R_{y} together form a cyclic peroxide comprising 4 to 8 links and comprising 3 or 4 oxygen atoms, Cj being one of the links of said cyclic peroxide, said cyclic peroxide being substituted with a group R₃, R₃ representing 1 to 8 groups which may be identical or different from each other, occupying any positions on the carbon atoms of the peroxide cycle. Such peroxide cycles may in particular consist of:
trioxanes with formula (XI):

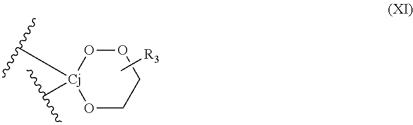

in which R₃ represents 1 to 4 groups, which may be identical or different, as defined for the compound with formula (I); or
trioxepanes with formula (XII):

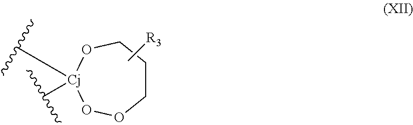

in which R₃ represents 1 to 6 groups, which may be identical or different, as defined for the compound with formula (I), or
trioxecanes with formula (XIII):

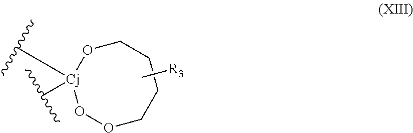

in which R₃ represents 1 or 8 groups, which may be identical or different, as defined for the compound with formula (I).

In formulae (XI), (XII) and (XIII) the carbon Cj is as defined for compounds with formula (I), i.e. Cj corresponds to the junction carbon between the cyclic peroxide and the cycle formed with the carbon Cj and radicals Z₁ and Z₂.

In formula (XI), R₃ advantageously represents 1 to 4 groups selected from hydrogen atoms and alkyl groups which may contain 1 to 10 carbon atoms, or two groups R₃ carried by the same carbon atom of the peroxide cycle together form a cycloalkyl group which may contain 3 to 7 carbon atoms or a bi- or tri-cyclic group which may contain 5 to 18 carbon atoms.

Compounds in accordance with the invention which may also be cited include a sixth group of compounds which have formula (I.2) below:

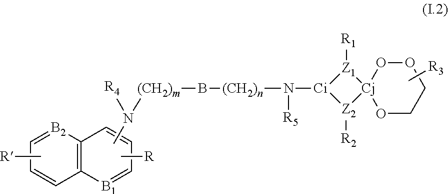

in which R, R′, B₁, B₂ and R₄ are as defined for the compound with formula (IIa) and B, Z₁, Z₂, Ci, Cj, R₁, R₂, R₃, R₅, m and n are as defined for the compound with formula (I).

Compounds in accordance with the invention which may also be cited include a seventh group of compounds which have formula (I.3) below:

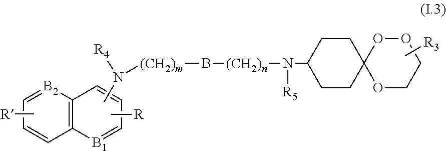

in which R, R′, B₁, B₂ and R₄ are as defined for the compound with formula (IIa) and B, R₃, R₅, m and n are as defined for the compound with formula (I).

Compounds in accordance with the invention which may be cited include compounds with formulae (1.1), (1.2) and (1.3) in which B represents a group selected from:

cis-1,2-methylenecyclopentyl, trans-1,2-cyclohexyl, cis-1,2-cyclohexyl, cis-1,2-methylenecyclohexyl, trans-1,4-cyclohexyl, cis-1,4-cyclohexyl, a cis/trans-1,4-cyclohexyl mixture, a cis/trans-1,3-cyclohexyl mixture, a cis/trans-1,3-dimethylenecyclohexyl mixture, cis-1,4-dimethylenecyclohexyl and 4,4′-methylene-bis-cyclohexane.

Compounds in accordance with the invention which may be cited include an eighth group of compounds which have formula (I) in which:

A represents an aminoquinoline with formulae (IIb) or (IIc) below:

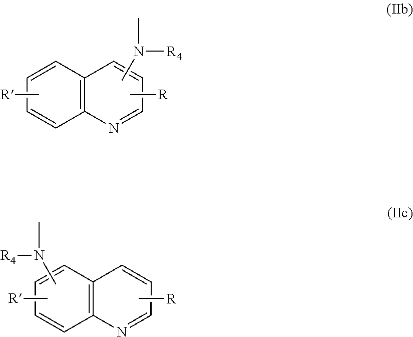

in which R, R′ and R₄ are as defined for the compound with formula (IIa);

B represents a group selected from:
a cycloalkyl group which may contain 3 to 8 carbon atoms, optionally substituted with one or more groups selected from: a halogen atom, a hydroxyl group, an alkyl group which may contain 1 to 6 carbon atoms or a cycloalkyl group which may contain 3 to 6 carbon atoms;
or B represents 2 cycloalkyl groups which may contain 3 to 6 carbon atoms, said cycloalkyls being connected together via a single bond or an alkylene chain which may contain 1 or 2 carbon atoms;

m and n independently represent 0, 1 or 2;

R₅ represents a hydrogen atom;

Z₁ and Z₂, which may be identical or different, represent an alkylene radical which may contain 1 to 4 saturated or unsaturated carbon atoms, the entity Z₁+Z₂+Ci+Cj thus representing:
either a cycloalkyl group which may contain 3 to 10 carbon atoms;
or a poly-cyclic structure which may contain 4 to 18 carbon atoms; Z₁ or Z₂ possibly representing a single bond between the carbon atoms Ci and Cj, it being understood that Z₁ and Z₂ cannot both represent a single bond at the same time;

R₁ and R₂ represent a hydrogen atom;

Rₓ and R_{y} together form a cyclic peroxide comprising 4 to 8 links and comprising 1 or 2 supplemental oxygen atoms in the cyclic structure (i.e. a total of 3 or 4 oxygen atoms in the cycle), Cj being one of the vertices of said cyclic peroxide;

said cyclic peroxide being substituted with a group R₃, R₃ representing 1 to 8 identical or different groups, occupying any positions on the carbon atoms of the peroxide cycle and being selected from the following atoms and groups:
hydrogen, halogen, an —OH, —CF₃, —NO₂, —OCF₃, aryl, —O-aryl, heteroaryl, alkyl or —O-alkyl group, said alkyl groups containing 1 to 10 carbon atoms;
or two groups R₃ carried by the same carbon atom of the peroxide cycle may together form a cycloalkyl group which may contain 3 to 7 carbon atoms or a bi- or tri-cyclic group which may contain 4 to 18 carbon atoms (which will thus be in the spiro position on the peroxide cycle).
   Compounds with formula (I) in accordance with the invention which may be cited include a ninth group of compounds selected from:

### PA1103, PA1265, PA1251, PA1252, PA1253, PA1255, PA1271, PA1269, PA1259, PA1258, PA1256, PA1268, PA1260, PA1188, PA1261, PA1207, PA1262, PA1263 and PA1264.

Finally, compounds with formula (I) in accordance with the invention which may be cited include a tenth group of compounds selected from:

### PA1305, PA1308, PA1329, PA1333, PA1335, PA1278, PA1279, PA1280, PA1286, PA1330, PA1331, PA1332 and PA1336.

The references shown above refer to the compounds in the examples below.

The invention also concerns a process for preparing a compound with formula (I).

In accordance with the invention, to prepare the compound with formulae (I), a compound with formula (III) as follows:

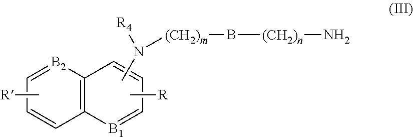

in which B, R, R′, B₁, B₂ and R₄ are as defined for the compound with formula (IIa) and B, m and n are as defined for the compound with formula (I), is reacted with a compound with formula (II) below:

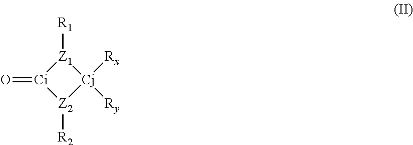

in which R₁, R₂, Z₁, Z₂, Rₓ and R_{y} are as defined in the compounds with formula (I).

The ketone and the primary amine are coupled in the presence of a reducing agent such as sodium cyanohydroboride, at ambient temperature, and an alcoholic solvent such as methanol, isopropanol or an alcohol mixture.

Said compounds are, for example, employed in an amine/primary ketone molar ratio of about 1.5, the reducing agent being used in an amount of 0.7 equivalent/ketone.

Compounds with formula (III) are obtained, for example, by reacting a compound with formula (V) as follows:

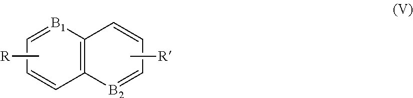

in which B₁, B₂, R and R′ are as defined in the compound with formula (IIa), it being understood that at least R or R′ represents a halogen atom; with a diamine with formula (IV) below:

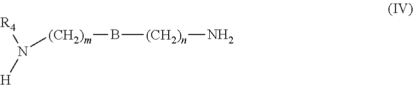

In general, peroxide derivatives with formula (II) comprising residues Rₓ and R_{y}, may be synthesized by analogy with the techniques presented in the work by S. Pataï: “The Chemistry of Peroxides”, John Wiley and Sons Ltd, 1983.

Compounds with formula (II) may also be obtained by reacting a triethylsilyldioxy alcohol or a suitable hydroperoxy alcohol with a diketone such as 1,4-cyclohexadione with formula (XX) or cis-bicyclo[3.3.0]octane-3,7-dione with formula (XXI):

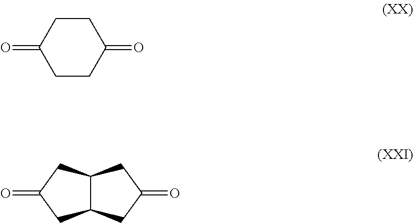

to produce trioxane derivatives with general formula (IIbis):

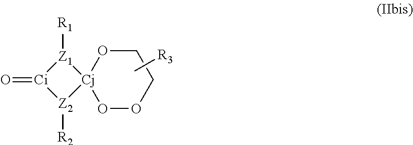

in which Z₁, Z₂, R₁, R₂ Ci, Cj and R₃ are as defined for the compound with formula (I).

These trioxanes are obtained by reacting a triethylsilyldioxy alcohol or a suitable hydroperoxy alcohol with a diketone, preferably in an amount of 3 molar equivalents of diketone. The reaction is, for example, carried out in the presence of para-toluenesulphonic acid, at ambient temperature for 30 minutes. The functionalized trioxane is then purified. Column chromatography may be used, for example.

Coupling of a compound with formula (III) with a compound with formula (II) is followed, as appropriate, by a reaction with a pharmaceutically acceptable acid, to obtain the coupling product in the salt form. To this end, basic nitrogens are protonated by adding a pharmaceutically acceptable organic or mineral acid. The reaction may be carried out with 2 equivalents of acid. The protonated product is then recovered and undergoes one or more purification steps if necessary.

The starting compounds and the reagents, when their implementation is not described, are commercially available or described in the literature, or may be prepared using methods which have been described in the literature or which are known to the skilled person.

The following examples describe the preparation of certain compounds which are in accordance with the invention. These examples are non-limiting and are given purely by way of illustration of the present invention.

In the following:

Me=methyl;
Et=ethyl;
HPLC=high pressure liquid chromatography;
min=minute(s)

### 1—Synthesis of PA 1103, (Figure 1)

### N-(7-chloro-quinolin-4-yl)-N′-(3,3-dimethyl-1,2,5-trioxa-spiro[5.5]undec-9-yl)-cyclohexane-trans-1,4-diamine

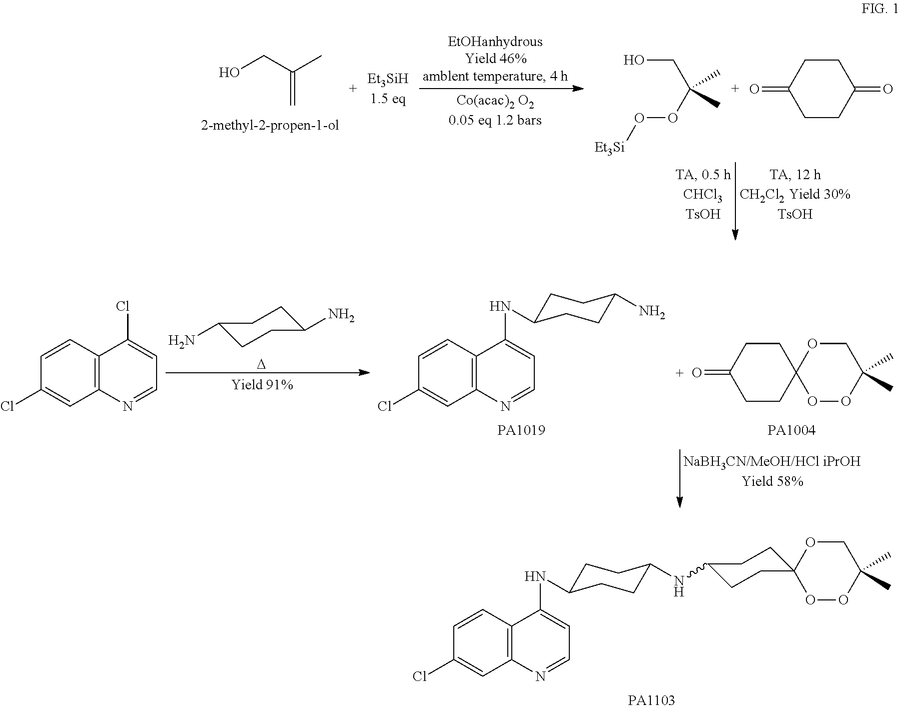

### 1-1: Synthesis of 3-methyl-3-[(triethylsilyl)dioxy]-butanol 1

The method followed was that described by P. M. O'Neill et al., (Tetrahedron Letters, 42, 2001, 4569-4571)

### 1-2: Synthesis of 3,3-dimethyl-1,2,5-trioxa-spiro[5.5]undecan-9-one

PA1004

7.84 g (35 mmoles) of 3-methyl-3-[(triethylsilyl)dioxy]-butanol 1 and 11.96 g (106 mmoles) of 1,4-cyclohexanedione was dissolved in 200 ml of chloroform. 4.66 g (24 mmoles) of para-toluenesulphonic acid was added, under argon at ambient temperature, and the mixture was stirred for 30 minutes. The reaction medium was then purified directly by chromatography (SiO₂ 60ACC 70-200 μm, eluent: CH₂Cl₂, ether (95/5, v/v)). The solvents for the phases containing PA1004 were evaporated off and 2.13 g (Yield=30%) of the compound was obtained in the form of a white solid.

Melting point: 71° C.

### 1-3: Synthesis of N-(7-chloro-quinolin-4-yl)-cyclohexane-trans-1,4-diamine

PA1019

58 g (0.29 mole) of 4,7-dichloroquinoline and 100 g (0.87 mole) of trans-1,4-diaminocyclohexane were heated to 135° C. for 1 h45 then the mixture was heated to 190° C. for 45 minutes. When the reaction medium had solidified, heating was stopped and the mixture was allowed to return to ambient temperature. 300 ml of 1M NaOH was added to the reaction medium and a precipitate formed. The medium was filtered and the precipitate washed with 1 l of distilled water. The precipitate was dried and used without further purification in the next step: 73 g (Yield=91%). Production of PA1019 followed the purification protocol described below: 3 g of the impure product was dissolved in 10 ml of CH₂Cl₂ then 50 ml of n-hexane was added and the mixture was filtered. The precipitate obtained was dissolved with heat in a minimum of ethyl acetate then poured onto 5 times the volume of n-hexane and filtered. PA1019 was obtained in the form of a beige powder (Yield=37%).

Melting point: 174° C.

### 1-4: Synthesis of PA1103

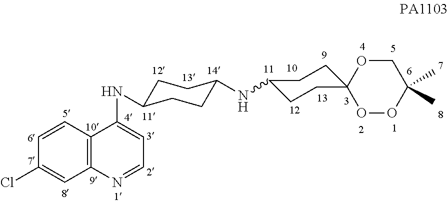

PA1019 (4.9 g; 18 mmoles) was dissolved in 120 ml of MeOH, then 2.4 ml of 5.5M HCl in isopropanol was added under argon at ambient temperature. 2.4 g (12 mmoles) of ketone PA1004 was added and the mixture was stirred for 1 h. NaBH₃CN (0.53 g; 8.4 mmoles) dissolved in 25 ml of MeOH was then added to the mixture, with stirring and under argon. The mixture was stirred at ambient temperature for 24 hours. 200 ml of distilled water then 200 ml of CH₂Cl₂ were added to the reaction medium and the organic phase was extracted, adding a further 200 ml of CH₂Cl₂. This organic phase was dried over Na₂SO₄, filtered and the solvents were evaporated off. The impure product obtained was purified by flash chromatography on a silica column (eluent: CH₂Cl₂/Et₃N, gradient: 10 min: CH₂Cl₂/Et₃N 98/2, v/v; 10 to 60 min: CH₂Cl₂/Et₃N 98/2, v/v to CH₂Cl₂/Et₃N, 90/10, v/v; 60 to 90 min CH₂Cl₂/Et₃N, 90/10, v/v). The phases containing PA1103 were combined, evaporated and the impure product was re-dissolved with heat in 400 ml of ethyl acetate and 400 ml of distilled water. This organic phase was washed with 200 ml of distilled water, dried over Na₂SO₄, filtered and evaporated off. 3.2 g (Yield=58%) of the compound PA1103 was obtained in the form of a powder.

Melting point: 176° C. (deg).

¹H NMR (250 MHz, 298K, CDCl₃): δ, ppm: 8.49 (d, J=5.4 Hz, 1H, HC2′), 7.93 (d, J=1.9 Hz, 1H, HC8′), 7.62 (d, J=9.0 Hz, 1H, HC5′), 7.32 (dd, J=9.0 Hz, J=2.2 Hz 1H, HC6′), 6.40 (d, J=5.5 Hz, 1H, HC3′), 4.87 (d, J=7.2 Hz, 1H, NH), 3.90-3.20 (m, 2H+1H, HC5+HC11′), 2.90-2.50 (m, 1H+1H+1H, HC14′+HC11+HCcyclohexyl), 2.25-1.18 (m, 15H+1H+3H, HCcyclohexyl+NH+HC7.8), 1.09 (broad s, 3H, HC7.8). MS (DCl/NH₃>0) m/z (%): 460 (MH⁺, 100%).

### 1-5: Separation of Two Isomers of PA1103

The two isomers of PA1103 were separated by supercritical HPLC chromatography: Berger Prep SFC supercritical chromatography system. (Chiral phase: CHIRALPAK AD-H 5 μm, Mobile phase: CO₂/Polar modifier=ethanol (60%/40%) (% by volume)). About 605 mg of PA1103 was dissolved with ultrasound in about 25 ml of ethanol then purified by supercritical HPLC chromatography. 116 mg of the first isomer PA1249 and 127 mg of the second isomer PA1250 were recovered.

PA1249:

Melting point: 176° C. (deg).

¹H NMR (400 MHz, 298K, CDCl₃): δ, ppm: 8.54 (d, J=5.2 Hz, 1H, HC2′), 7.97 (d, J=2 Hz, 1H, HC8′), 7.64 (d, J=8.8 Hz, 1H, HC5′), 7.37 (dd, J=8.8 Hz, J=2 Hz, 1H, HC6′), 6.45 (d, J=5.2 Hz, 1H, HC3′), 4.82 (d, J=6.8 Hz, 1H, NH), 3.77-3.49 (m, 2H+1H, HC5+HC11′), 2.90-2.69 (m, 1H+1H+1H, HC14′+HC11+HCcyclohexyl), 2.29-1.25 (m, 15H+1H+3H, HCcyclohexyl+NH+HC7.8), 1.15 (broad s, 3H, HC7.8). LCMS (MeOH>0) m/z (%): 460.2 (MH⁺, 100%).

PA1250:

Melting point: 175° C. (deg).

¹H NMR (400 MHz, 298K, CDCl₃): δ, ppm: 8.53 (d, J=5.4 Hz, 1H, HC2′), 7.97 (d, J=2 Hz, 1H, HC8′), 7.64 (d, J=8.8 Hz, 1H, HC5′), 7.37 (dd, J=8.8 Hz, J=2 Hz, 1H, HC6′), 6.45 (d, J=5.2 Hz, 1H, HC3′), 4.82 (d, J=7.2 Hz, 1H, NH), 3.84-3.47 (m, 2H+1H, HC5+HC11′), 2.94-2.70 (m, 1H+1H+1H, HC14′+HC11+HCcyclohexyl), 2.28-1.23 (m, 15H+1H+3H, HCcyclohexyl+NH+HC7.8), 1.11 (broad s, 3H, HC7.8). LCMS (MeOH>0) m/z (%): 460.2 (MH⁺, 100%).

By way of example, the corresponding salts (1a., 1b. and 1c.) of PA1103 were synthesized.

1a.—Synthesis of di-phosphate salt of PA1103 (PA1278):

The compound PA1103 obtained above (403 mg; 0.88 mmole) was dissolved in 5 ml of EtOH at 30° C. then 1.2 ml of a solution of 405 mg of 85% phosphoric acid (H₃PO₄) in 2 ml of EtOH was added. After stirring for 30 minutes at ambient temperature, the precipitate was drained, washed with 1.5 ml of EtOH then vacuum dried at 45° C.

1b.—Synthesis of di-acetate salt of PA1103 (PA1279):

The compound PA1103 (388 mg; 0.84 mmole) was dissolved in 4 ml of THF at ambient temperature then 1.1 ml of a solution of 200 mg of AcOH in 2 ml of THF was added. After stirring for 1 h15 at ambient temperature, the precipitate was drained, washed with 0.5 ml of THF and dried in air.

1c.—Synthesis of di-sulphate salt of PA1103 (PA1280):

The compound PA1103 (360 mg; 0.78 mmole) was dissolved in 4.5 ml of EtOH then 1 ml of a solution of 310 mg of H₂SO₄ in 2 ml of EtOH was slowly added. After stirring for 3 h at ambient temperature, the precipitate was drained then vacuum dried at 45° C.

### 2—Synthesis of PA 1265, (Figure 2)

### N-(7-chloro-quinolin-4-yl)-M-(3,3-dimethyl-1,2,5-trioxa-spiro[5.5]undec-9-yl)-cyclohexane-cis-1,4-diamine

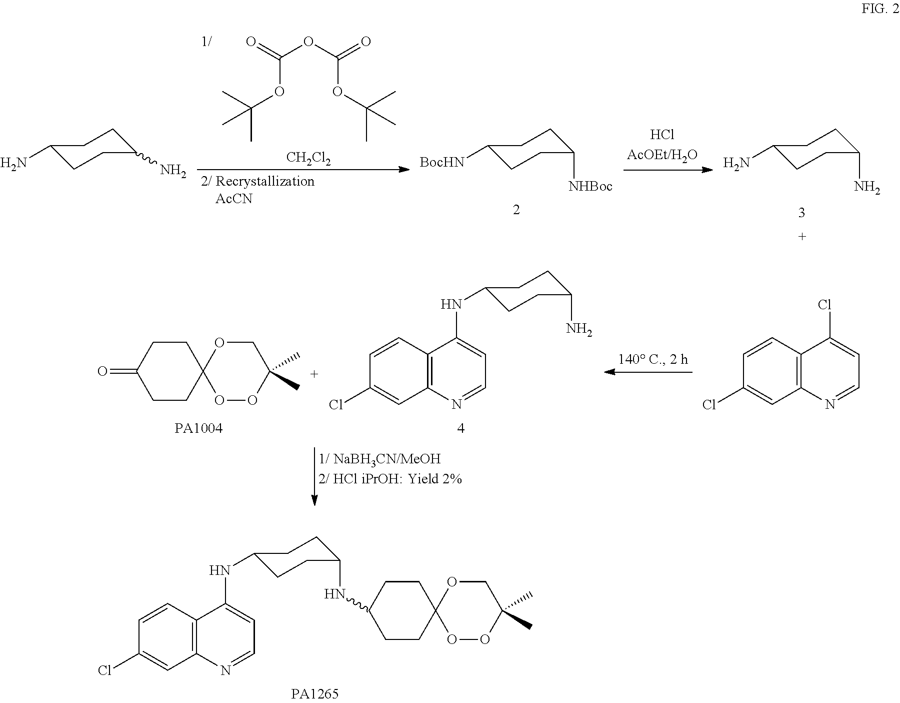

### 2-1: Synthesis of tert-butyl ester of (cis-4-tert-butoxycarbonylamino-cyclohexyl)-carbamic acid 2

5 g (43 mmoles) of a commercial cis/trans-1,4-cyclohexane diamine mixture was dissolved in 50 ml of CH₂Cl₂. 18.8 g (86 mmoles) of di-tert-butyl dicarbonate which had been dissolved in 50 ml of CH₂Cl₂ was added dropwise. The mixture was stirred overnight at ambient temperature. 500 ml of distilled water and 200 ml of CH₂Cl₂ were added and the organic phase was extracted then dried over Na₂SO₄ and filtered. The solvents were evaporated off, producing a white powder: 11.3 g (Yield=83%). This 11.3 g of mixed cis/trans-(4-tert-butoxycarbonylamino-cyclohexyl)-carbamic acid tert-butyl ester was dissolved in 100 ml of acetonitrile. The mixture was refluxed for 20 min then filtered. The filtrate was cooled in an ice bath for 1 h and a precipitate appeared. Next, the mixture was filtered. 4.8 g (Yield=36%) of cis-(4-tert-butoxycarbonylamino-cyclohexyl)-carbamic acid tert-butyl ester 2 was obtained in the form of a white powder.

Melting point: 144° C. (deg).

### 2-3: Synthesis of cis-1,4-cyclohexane diamine 3

4.8 g (15 mmoles) of cis-(4-tert-butoxycarbonylamino-cyclohexyl)-carbamic acid tert-butyl ester 2 was dissolved in 50 ml of ethyl acetate and 25 ml of 3M HCl in ethyl acetate was added to the mixture. The mixture was stirred overnight at ambient temperature. The pH was then swung by adding 15 g of NaOH and the aqueous phase was extracted with 300 ml of CH₂Cl₂. The organic phase was dried over Na₂SO₄ and filtered. The solvents were evaporated off. 1.7 g (quant) of the compound was obtained in the form of a colourless oil.

### 2-4: Synthesis of N-(7-chloro-quinolin-4-yl)-cis-1,4-cyclohexane-diamine 4

0.81 g (4.1 mmoles) of 4,7-dichloroquinoline and 1.4 g (12 mmoles) of cis-1,4-diaminocyclohexane were heated to 135° C. for 1 h45 then the mixture was, heated to 190° C. over 45 min. When the reaction medium had solidified, heating was stopped and the mixture was allowed to return to ambient temperature. 10 ml of 1M NaOH was added. The reaction medium was stirred overnight. The aqueous phase was withdrawn and the impure product was dissolved in 5 ml of methanol; next, 50 ml of diethyl ether was added. The precipitate formed was filtered, re-dissolved in 1 ml of CH₂Cl₂ then 100 ml of n-hexane was added. After filtering, 0.5 g (Yield=44%) of compound 4 was obtained in the form of a beige powder.

### 2-5: Synthesis of PA1265

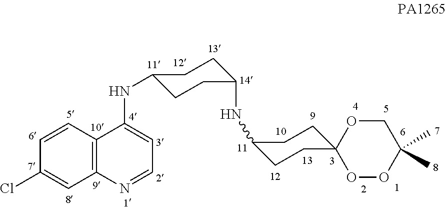

Compound 4 (0.5 g; 1.8 mmoles) was dissolved in 20 ml of MeOH then 0.2 ml of 5.5M HCl in isopropanol was added under argon at ambient temperature. 0.24 g (1.2 mmoles) of ketone PA1004 was added and the mixture was stirred for 1 h. NaBH₃CN (53 mg; 0.8 mmole) was added to the mixture, with stirring and under argon. The reaction medium was stirred at ambient temperature for 24 hours. The solvents were vacuum evaporated and the impure reaction product was purified by silica column chromatography (eluent: CH₂Cl₂/Et₃N, 90/10, v/v). The phases containing PA1265 were combined, evaporated and the impure product was re-dissolved in 100 ml of ethyl acetate. This organic phase was washed with 200 ml of distilled water, dried over Na₂SO₄, filtered and evaporated to produce an oil. This oil was precipitated by adding 1 ml of CHCl₃ and 50 ml of n-hexane to produce a powder identified as PA1265: 10 mg (Yield=2%).

Melting point: 140° C. (deg).

¹H NMR (250 MHz, 298K, CDCl₃): δ, ppm: 8.48 (d, J=5.4 Hz, 1H, HC2′), 7.92 (d, J=1.9 Hz, 1H, HC8′), 7.71 (d, J=8.9 Hz, 1H, HC5′), 7.32 (dd, J=8.9 Hz, J=2.1 Hz 1H, HC6′), 6.39 (d, J=5.5 Hz, 1H, HC3′), 5.16 (t, J=6.2 Hz, 1H, NH), 3.90-3.20 (m, 2H+1H, HC5+HC11′), 2.87-2.21 (m, 1H+1H+1H, HC14′+HC11+HCcyclohexyl), 2.25-1.18 (m, 15H+3H, HCcyclohexyl+HC7.8), 1.09 (broad s, 3H, HC7.8). MS (DCl/NH3>0) m/z (%): 460 (MH⁺, 100%).

### 3—Synthesis of PA 1251, (Figure 3)

### N-(2,8-bis-trifluoromethyl-quinolin-4-yl)-N′-(3,3-dimethyl-1,2,5-trioxa-spiro[5.5]undec-9-yl)-cyclohexane-trans-1,4-diamine

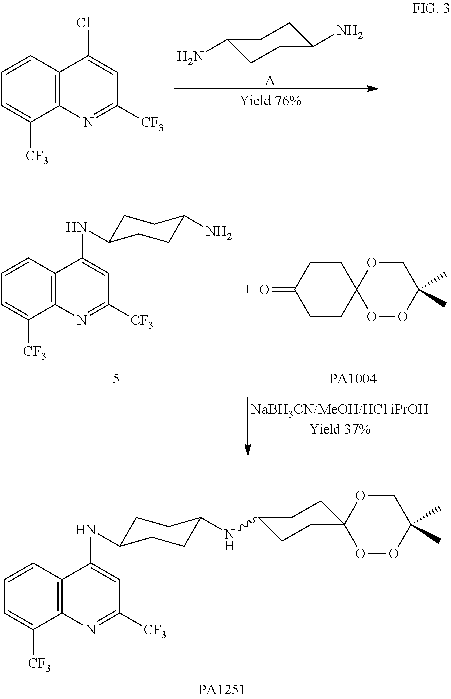

### 3-1: Synthesis of trans-N-(2,8-bis-trifluoromethyl-quinolin-4-yl)-cyclohexane-1,4-diamine 5

2 g (6.6 mmoles) of 4-chloro-2,8-bis(trifluoromethyl)quinoline and 2.3 g (20 mmoles) of trans-1,4-diaminocyclohexane were heated to 155° C. for 2 h. The mixture was allowed to return to ambient temperature and 13 ml of 1M NaOH was added to the reaction medium to produce a precipitate. The medium was filtered and the precipitate was washed with 2×20 ml of distilled water. The impure product was dissolved in 30 ml of CH₂Cl₂ then 50 ml of n-hexane was added and the mixture was filtered. The precipitate obtained was dissolved in 100 ml of CH₂Cl₂, the organic phase was washed with 100 ml of distilled water and dried over Na₂SO₄, filtered and evaporated. 1.9 g (Yield=76%) of compound 5 was obtained in the form of a powder. MP: 173° C.

### 3-2: Synthesis of PA1251

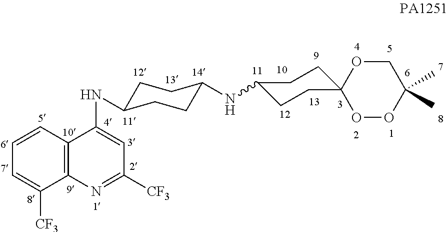

Compound 5 (0.56 g, 1.5 mmoles) was dissolved in 12 ml of MeOH then 0.2 ml of 5.5M HCl in isopropanol was added under argon at ambient temperature. 0.20 g (1 mmole) of the ketone PA1004 was then added and the mixture was stirred for 1 h. NaBH₃CN (44 mg, 0.8 mmole) which had been dissolved in 2 ml of MeOH was added to the mixture, with stirring and under argon. The reaction medium was stirred at ambient temperature for 24 hours. The solvents were vacuum evaporated and the impure reaction product was purified by silica column chromatography (eluent: CH₂Cl₂/Et₃N, 95/5, v/v). The phases containing PA1251 were combined, evaporated and the impure product was re-dissolved in 100 ml of ethyl acetate. This organic phase was washed with 200 ml of distilled water, dried over Na₂SO₄, filtered and evaporated off. The powder obtained was ground and 15 ml of n-hexane was added. This suspension was filtered and 1 ml of ether was added, then the mixture was vacuum evaporated. 0.21 g (Yield=37%) of compound PA1251 was obtained in the form of a powder.

Melting point: 168° C. (deg).

¹H NMR (250 MHz, 298K, CDCl₃): δ, ppm: 8.04 (d, J=7.2 Hz, 1H, HC7′), 7.91 (d, J=8.4 Hz, 1H, HC5′), 7.53 (dd, J=7.8 Hz, J=7.9 Hz, 1H, HC6′), 6.77 (s, 1H, HC3′), 5.09 (d, J=7.3 Hz, 1H, NH), 3.90-3.30 (m, 2H+1H+1H, HC5+HC11′+HC11), 2.90-2.50 (m, 1H+1H+1H, HC14′+HCcyclohexyl), 2.27-1.20 (m, 14H+1H+3H, HCcyclohexyl+NH+HC7.8), 1.10 (broad s, 3H, HC7.8). MS (DCI/NH₃>0): m/z (%): 562 (MH⁺, 100%).

### 4—Synthesis of PA 1252, (Figure 4)

### N-(3,3-dimethyl-1,2,5-trioxa-spiro[5.5]undec-9-yl)-N′-(7-trifluoromethyl-quinolin-4-yl)-cyclohexane-trans-1,4-diamine

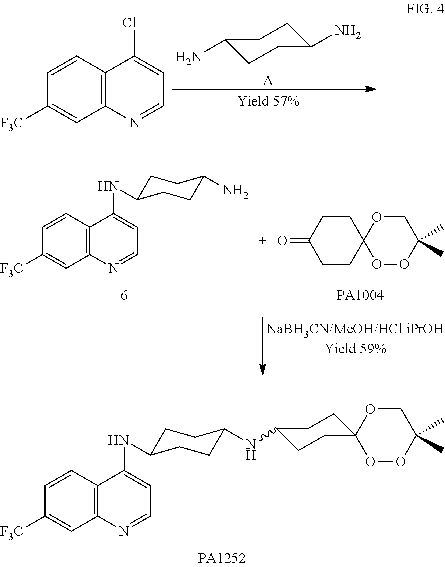

### 4-1: Synthesis of trans-N-(7-trifluoromethyl-quinolin-4-yl)-cyclohexane-1,4-diamine 6

10 g (43 mmoles) of 4-chloro-7-trifluoromethylquinoline and 14.8 g (129 mmoles) of trans-1,4-diaminocyclohexane were heated to 130° C. for 1 h, then the mixture was heated to 190° C. for 1 h; the mixture was allowed to return to ambient temperature. 85 ml of 1M NaOH was added to the reaction medium to produce a precipitate. The medium was filtered and the precipitate was washed with 250 ml of distilled water. The impure product was dissolved in 100 ml of CH₂Cl₂ then 900 ml of n-hexane was added and the mixture was filtered; the precipitate obtained was re-dissolved in 500 ml of CH₂Cl₂, the mixture was filtered, the organic phase was recovered and washed with 750 ml of distilled water then dried over Na₂SO₄, filtered and concentrated to a volume of 100 ml. 900 ml of n-hexane was poured onto this impure product and a precipitate appeared. The precipitate was filtered and dried. 4.8 g (yield=36%) of compound 6 was obtained in the form of a powder. MP: 186.5° C.

### 4-2: Synthesis of PA1252

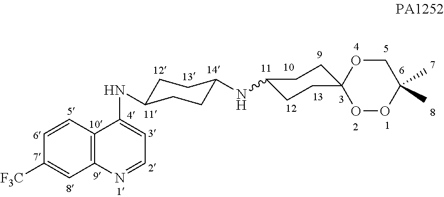

Compound 6 (0.46 g, 1.5 mmoles) was dissolved in 12 ml of MeOH then 0.2 ml of 5.5M HCl in isopropanol under argon was added at ambient temperature. 0.20 g (1 mmole) of ketone PA1004 was added and the mixture was stirred for 1 h. NaBH₃CN (44 mg; 0.8 mmole) which had been dissolved in 2 ml of MeOH was then added to the mixture, with stirring and under argon. The reaction medium was stirred at ambient temperature for 24 hours. The solvents were vacuum evaporated and the impure reaction product was purified by silica column chromatography (eluent: CH₂Cl₂/Et₃N, 95/5, v/v). The phases containing PA1252 were combined, evaporated and the impure product was re-dissolved in 100 ml of ethyl acetate. This organic phase was washed with 200 ml of distilled water, dried over Na₂SO₄, filtered and evaporated off. 0.29 g (Yield=59%) of compound PA1252 was obtained in the form of a powder.

Melting point: 166.5° C. (deg).

¹H NMR (250 MHz, 298K, CDCl₃): δ, ppm: 8.60 (d, J=5.4 Hz, 1H, HC2′), 8.24 (s, 1H, HC8′), 7.79 (d, J=8.7 Hz, 1H, HC5′), 7.56 (dd, J=8.9 Hz, J=1.7 Hz, 1H, HC6′), 6.51 (d, J=5.4 Hz, 1H, HC3′), 4.87 (d, J=7.2 Hz, 1H, NH), 3.90-3.30 (m, 2H+1H, HC5+HC11′), 2.90-2.50 (m, 1H+1H+1H, HC14′+HC11+HCcyclohexyl), 2.27-1.20 (m, 15H+1H+3H, HCcyclohexyl+NH+HC7.8), 1.10 (broad s, 3H, HC7.8). MS (DCl/NH₃>0): m/z (%): 494 (MH⁺, 100%).

### 5—Synthesis of PA 1253, (Figure 5)

### N-(3,3-dimethyl-1,2,5-trioxa-spiro[5.5]undec-9-yl)-N′-(6-dimethylamino-quinolin-4-yl)-cyclohexane-trans-1,4-diamine

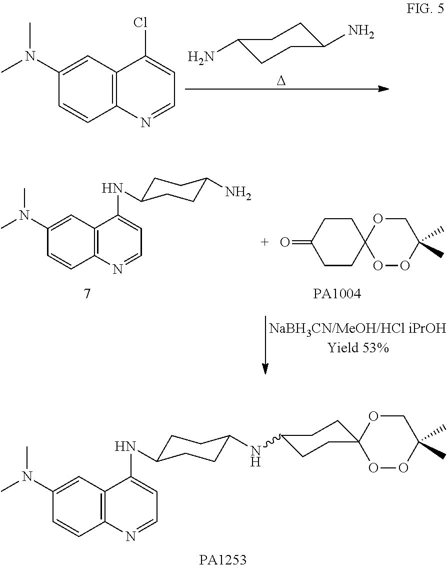

### 5-1: Synthesis of trans-N-(6-N,N-dimethyl-quinolin-4-yl)-cyclohexane-1,4-diamine 7

1.5 g (7.3 mmoles) of 4-chloro-6-dimethylaminoquinoline (prepared using the method described by Riegel et al., J. Am. Chem. Soc., 1946, 68, 1264) and 2.5 g (22 mmoles) of trans-1,4-diaminocyclohexane were heated to 130° C. for 2 h then to 190° C. for 9 h. The mixture was allowed to return to ambient temperature and 15 ml of 1M NaOH was added to the reaction medium to produce an oil. The oil was washed with 10 ml of distilled water and 20 ml of CH₂Cl₂ was added. The organic phase was decanted, washed with 3×20 ml of distilled water then dried over Na₂SO₄, filtered and concentrated to a volume of 2 ml. 100 ml of n-hexane was then poured onto the impure product and a precipitate appeared. This precipitate was filtered and dried. 0.5 g (Yield=24%) of compound 7 was obtained in the form of a powder.

### 5-2: Synthesis of PA1253

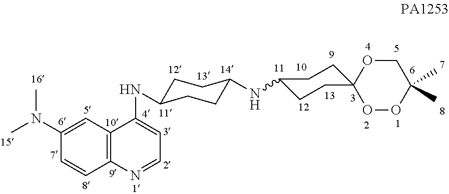

Compound 7 (0.43 g, 1.5 mmoles) was dissolved in 12 ml of MeOH then 0.2 ml of 5.5M HCl in isopropanol was added under argon at ambient temperature. 0.20 g (1 mmole) of ketone PA1004 was then added and the mixture was stirred for 1 h. NaBH₃CN (44 mg; 0.8 mmole) which had been dissolved in 2 ml of MeOH was then added to the mixture. The reaction medium was stirred and kept under argon at ambient temperature for 24 hours. The solvents were vacuum evaporated and the impure product was purified by silica column chromatography (eluent: CH₂Cl₂/Et₃N, 95/5, v/v). The phases containing PA1253were combined, evaporated and the impure product was dissolved in 100 ml of ethyl acetate. This organic phase was washed with 200 ml of distilled water, dried over Na₂SO₄, filtered and evaporated off. 0.25 g (Yield=53%) of compound PA1253 was obtained in the form of a powder.

Melting point: 193° C. (deg).

¹H NMR (250 MHz, 298K, CDCl₃): δ, ppm: 8.35 (d, J=5.2 Hz, 1H, HC2′), 7.86 (d, J=9.3 Hz, 1H, HC8′), 7.30 (dd, J=9.3 Hz, J=2.6 Hz, 1H, HC7′), 6.53 (d, J=2.6 Hz, 1H, HC5′), 6.38 (d, J=Hz, 1H, HC3′), 4.56 (d, J=7.1 Hz, 1H, NH), 3.90-3.30 (m, 2H+1H, HC5+HC11′), 3.06 (s, 6H, HC15′+HC16′), 2.90-2.50 (m, 1H+1H+1H, HC14′+HC11+HCcyclohexyl), 2.27-1.20 (m, 15H+1H+3H, HCcyclohexyl+NH+HC7.8), 1.10 (broad s, 3H, HC7.8). MS (DCI/NH₃>0): m/z (%): 469 (MH⁺, 33%).

### 6—Synthesis of PA 1255, (Figure 6)

### N-(7-chloro-quinolin-4-yl)-N′-(3,4-dimethyl-1,2,5-trioxa-spiro[5.5]undec-9-yl)-cyclohexane-trans-1,4-diamine

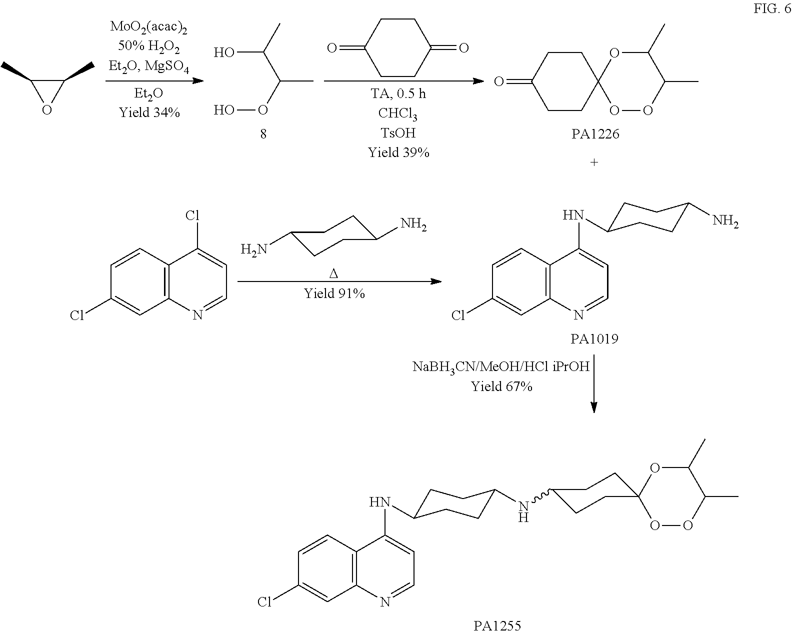

### 6-1: Synthesis of 3-hydroperoxy-butan-2-ol 8

150 ml of ether and 8.3 ml (147 mmoles) of 50% H₂O₂ in solution in water were mixed in an Erlenmeyer flask. 10 g (83 mmoles) of anhydrous MgSO₄ was added in small quantities. The mixture was stirred for 20 min then filtered through a frit. The filtrate was poured into a 500 ml flask containing a mixture of 10 ml of ether, 0.23 g (0.7 mmole) of MoO₂(acac)₂ and 1 g (14 mmoles) of cis-2.3-epoxybutane. The mixture was stirred at ambient temperature for 24 h. 100 ml of distilled water and 100 ml of ethyl acetate were added and the organic phase was extracted. The organic phase was washed with 100 ml of a saturated NaCl solution then dried over MgSO₄ and filtered. The solvents were evaporated off. 0.5 g (Yield=34%) of compound 8 was obtained in the form of a colourless oil.

### 6-2: Synthesis of 3,4-dimethyl-1,2,5-trioxa-spiro[5.5]undecan-9-one

PA1226

0.5 g (4.8 mmoles) of 3-hydroperoxy-butan-2-ol 8 and 1.61 g (14 mmoles) of 1,4-cyclohexanedione were dissolved in 50 ml of chloroform. 0.6 g (3.3 mmoles) of para-toluenesulphonic acid was added at ambient temperature under argon, and the mixture was stirred for 30 minutes. The reaction medium was then purified directly by chromatography (SiO₂ 60ACC 70-200 μm, eluent: CH₂Cl₂, ether (95/5, v/v)). The solvents for the phases containing PA1226 were evaporated off. 0.38 g (Yield=39%) of compound PA1226 was obtained in the form of a colourless oil.

### 6-3: Synthesis of PA1255

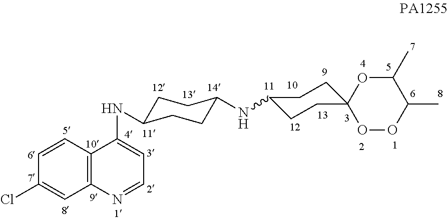

Compound PA1019 (0.8 g; 2.8 mmoles) was dissolved in 20 ml of MeOH then 0.4 ml of 5.5M HCl in isopropanol was added under argon at ambient temperature. 0.38 g (1.8 mmoles) of ketone PA1226 was added and the mixture was stirred for 1 h. NaBH₃CN (83 mg; 1.3 mmoles) was then added to the mixture, with stirring and under argon. The mixture was stirred at ambient temperature for 24 hours. The solvents were evaporated off and the reaction medium was purified by silica column chromatography (eluent: CH₂Cl₂/Et₃N, 80/20, v/v). The phases containing PA1255 were combined, evaporated off and the impure product was dissolved in 200 ml of ethyl acetate. This organic phase was washed with 200 ml of distilled water, dried over Na₂SO₄, filtered and evaporated. 0.58 g (Yield=67%) of compound PA1255 was obtained in the form of a powder.

Melting point: 166° C. (deg).

¹H NMR (250 MHz, 298K, CDCl₃): δ, ppm: 8.50 (d, J=5.4 Hz, 1H, HC2′), 7.94 (d, J=2.1 Hz, 1H, HC8′), 7.65 (d, J=9.1 Hz, 1H, HC5′), 7.34 (dd, J=8.9 Hz, J=2.1 Hz 1H, HC6′), 6.46 (d, J=5.5 Hz, 1H, HC3′), 4.93 (s, 1H, NH), 4.01-3.71 (m, 1H+1H, HC5+HC6), 3.48 (m, 1H, HC11′), 2.90-2.66 (m, 1H+1H+1H, HC14′+HC11+HCcyclohexyl), 2.24-1.20 (m, 15H+1H, HCcyclohexyl+NH), 1.14-1.06 (m, 6H, HC7.8). MS (DCl/NH₃>0) (%): 460 (MH⁺, 100%).

### 7—Synthesis of PA 1305, (Figure 7)

### N-(6-trifluoromethoxy-quinolin-4-yl)-N′-(3,3-dimethyl-1,2,5-trioxa-spiro[5.5]undec-9-yl)-cyclohexane-trans-1,4-diamine

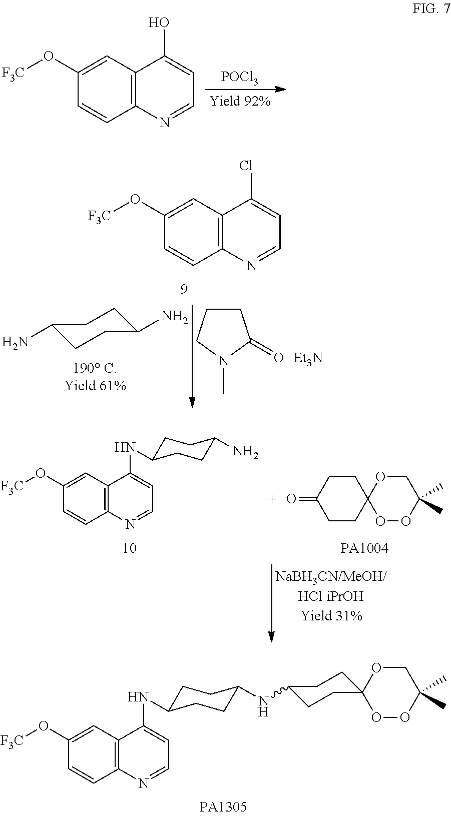

### 7-1: Synthesis of 4-chloro-6-trifluoromethoxy-quinoline 9

1 g (4.4 mmoles) of 6-trifluoromethoxy-quinolin-4-ol was dissolved in 4.1 ml (44 mmoles) of POCl₃. The mixture was heated to 115° C. for 3 h. After returning to ambient temperature, the POCl₃ was vacuum evaporated. 25 ml of distilled water then a few drops of NH₄OH were added to the residue obtained to bring the pH of the solution to a pH of about 8-9. The compound was extracted by adding 60 ml of CH₂Cl₂. The organic phase was dried over Na₂SO₄, filtered then the solvents were evaporated off to produce 0.9 g (Yield=92%) of a brown liquid identified as 9.

### 7-2: Synthesis of N-(6-trifluoromethoxy-quinolin-4-yl)-cyclohexane-1,4-diamine 10

0.5 g (2 mmoles) of 4-chloro-6-trifluoromethoxy-quinoline 9 was dissolved in 2 ml of N-methylpyrrolidinone. 0.7 g (6 mmoles) of trans-1,4-diaminocyclohexane and 280 μL (2 mmoles) of triethylamine were added to this solution; the mixture was then heated to 190° C. for 6 h30. The mixture was allowed to return to ambient temperature and 17 ml of 1M NaOH then 30 ml of ethyl acetate were added to the reaction medium. This mixture was heated to 50° C. and the organic phase was recovered. The extraction was repeated, adding 40 ml of distilled water to the aqueous phase obtained and 40 ml of ethyl acetate. The organic phases were then combined, dried with Na₂SO₄, filtered and the solvents were evaporated off to produce 0.4 g (Yield=61%) of 10.

### 7-3: Synthesis of PA1305

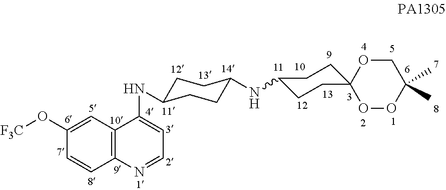

10 (0.35 g, 12 mmoles) in solution in 10 ml of MeOH was placed under argon at ambient temperature. 158 μl (0.8 mmole) of 5.5 M HCl in isopropanol and 0.16 g (0.8 mmole) of ketone PA1004 were added to this mixture. The mixture was stirred for 1 h. NaBH₃CN (0.035 g, 0.5 mmole) which had been dissolved in 2 ml of MeOH was then added to the mixture, with stirring and under argon. The medium was stirred at ambient temperature for 24 h. The mixture was then purified directly by silica column flash chromatography (eluent: ethyl acetate/Et₃N, gradient: 5 min; ethyl acetate/Et₃N 98/2, v/v; 5 to 45 min: ethyl acetate/Et₃N 98/2, v/v to ethyl acetate/Et₃N 90/10, v/v; 45 to 65 min ethyl acetate/Et₃N 90/10, v/v; 65 to 70 min: ethyl acetate/Et₃N 90/10, v/v to ethyl acetate/Et₃N 85/15, v/v; 70 to 95 min: ethyl acetate/Et₃N 85/15, v/v). The phases containing PA1305 were combined. This organic phase was washed with 200 ml of distilled water, dried over Na₂SO₄, filtered and evaporated. 0.19 g (Yield=31%) of compound PA1305 was obtained in the form of a powder.

Melting point: 165° C. (deg).

¹H NMR (250 MHz, 298K, CDCl₃): δ, ppm: 8.54 (d, J=5.4 Hz, 1H, HC2′), 7.99 (d, J=9.5 Hz, 1H, HC8′), 7.49-7.47 (m, 1H, HC7′+HC5′), 6.46 (d, J=2.3 Hz 1H, HC3′), 4.68 (d, J=7.1 Hz, 1H, NH), 3.90-3.20 (m, 2H+1H, HC5+HC11′), 2.90-2.50 (m, 1H+1H, HC11+NH), 2.23-1.15 (m, 17H+3H, HCcyclohexyl+HC7.8), 1.09 (broad s, 3H, HC7.8). MS (DCl/NH₃>0) m/z (%): 510 (MH⁺, 37%)_{.}

### 8—Synthesis of PA 1308, (Figure 8)

### N-(7-chloro-quinolin-4-yl)-M-(3,3-dimethyl-1,2,5-trioxa-spiro[5.5]undec-9-yl)-N′-methyl-cyclohexane-cis-1,4-diamine

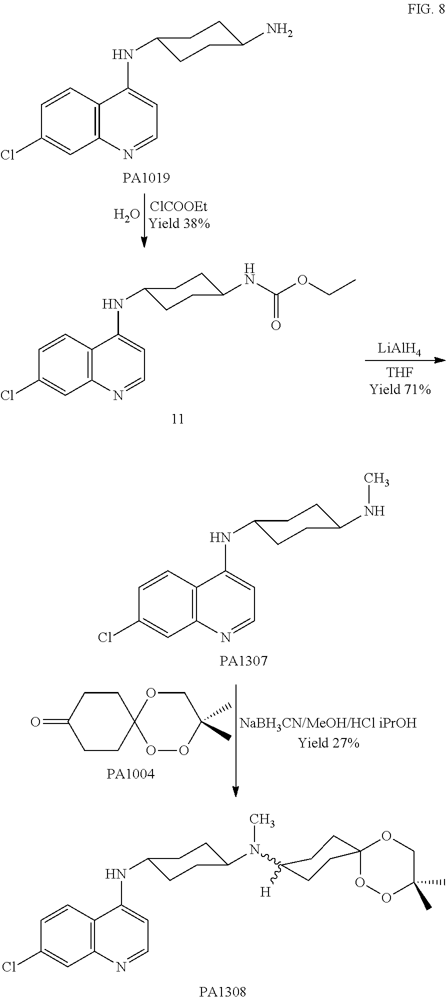

### 8-1: Synthesis of ethyl ester of [4-(7-chloro-quinolin-4-ylamino)-cyclohexyl]-carbamic acid 11

A suspension of 2.2 g (7.9 mmoles) of PA1019 was prepared in 400 ml of distilled water. 1.45 ml (11.9 mmoles) of ethylchloroformate was added dropwise at ambient temperature. The mixture was stirred overnight at ambient temperature. The pH of the mixture was then brought to a pH of 8 by adding NaHCO₃. The medium was then extracted with 1200 ml of CH₂Cl₂. The organic phase was dried over Na₂SO₄ and filtered. The solvents were evaporated to produce an impure product which was purified by silica column chromatography (eluent: ethyl acetate/Et₃N, 95/5, v/v). The phases containing 11 were washed with 500 ml of distilled water, dried over Na₂SO₄, filtered and evaporated to produce a powder identified as compound 11: 1.04 g (Yield=38%).

Melting point: 241° C. (deg).

### 8-2: Synthesis of N-(7-chloro-quinolin-4-yl)-N′-methyl-cyclohexane-1,4-diamine: PA1307

1 g (2.9 mmoles) of 11 was dissolved in 50 ml of dry THF. This solution was added dropwise, under argon, over one hour, to a solution, cooled in an ice bath, of 25 ml of dry THF and 11.5 ml (11.5 mmoles) of 1M LiAlH₄ in ether. When addition was complete, the mixture was refluxed for 30 min. Next, the reaction medium was hydrolysed and 250 ml of ethyl acetate was added. The recovered organic phase was dried over Na₂SO₄, filtered and evaporated to produce an impure product which was purified by silica column flash chromatography (eluent: CH₂Cl₂/MeOH/Et₃N, gradient: 5 min CH₂Cl₂/MeOH/Et₃N 90/9/1, v/v/v; 10 to 40 min: CH₂Cl₂/MeOH/Et₃N 90/9/1, v/v/v; to CH₂Cl₂/MeOH/Et₃N 80/18/2, v/v/v; 45 to 65 min: CH₂Cl₂/MeOH/Et₃N 80/18/2, v/v/v). The phases containing PA1307 were combined, evaporated and the impure product was dissolved in 500 ml of ethyl acetate and 250 ml of a solution of NaHCO₃ at a pH of 9. This organic phase was recovered, dried over Na₂SO₄, filtered and evaporated to produce a powder which was identified as PA1307: 0.59 g (Yield=71%).

Melting point: 185° C. (deg).

### 8-3: Synthesis of PA1308

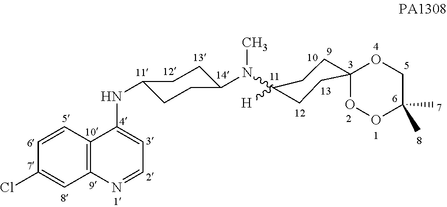

0.46 g (1.6 mmoles) of PA1307 was dissolved in 25 ml of MeOH then 210 μl (1.15 mmoles) of 5.5M HCl in isopropanol was added in argon at ambient temperature. 0.21 g (1.0 mmole) of ketone PA1004 was added and the mixture was stirred for 1 h. NaBH₃CN (46 mg, 0.7 mmole) was then added to the mixture, with stirring and under argon. The reaction medium was stirred at ambient temperature for 24 h. The solvents were vacuum evaporated and the impure reaction product was purified by silica column flash chromatography (eluent: ethyl acetate/Et₃N 95/5, v/v). The phases containing PA1308 were combined, washed with 200 ml of distilled water, dried over Na₂SO₄, filtered and evaporated to produce a powder identified as PA1308: 0.136 g (Yield=27%).

Melting point: 179° C. (deg).

¹H NMR (250 MHz, 298K, DMSOd₆): δ, ppm: 8.37-8.31 (m, 1H+1H, HC2′+HC5′), 7.75 (d, J=0.6 Hz, 1H, HC8′), 7.42 (dd, J=8.9 Hz, J=2.2 Hz 1H, HC6′), 6.92 (d, J=7.6 Hz, 1H, NH), 6.51 (d, J=5.6 Hz, 1H, HC3′), 3.90-3.40 (m, 2H+1H, HC5+HC11′), 2.57 (m, 1H+1H, HCcyclohexyl+HC11), 2.17 (s, 3H, H₃CN), 2.05-1.20 (m, 16H+3H, HCcyclohexyl+HC7.8), 1.05 (broad s, 3H, HC7.8). MS (DCl/NH3>0) m/z (%): 474 (MH⁺, 100%).

### 9—Synthesis of PA 1329, (Figure 9)

### N-(7-chloro-quinolin-4-yl)-N′-(3,3-dimethyl-1,2,5-trioxa-spiro[5.5]undec-9-yl)-N′-ethyl-cyclohexane-cis-1,4-diamine

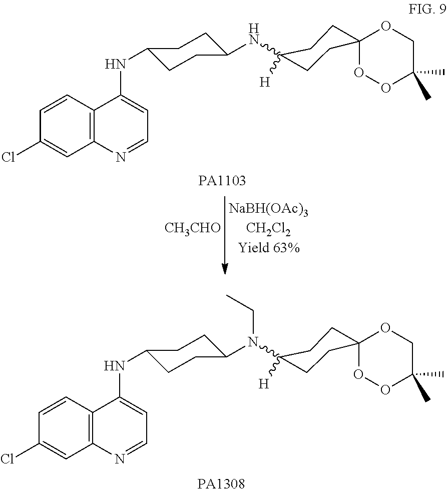

### 9-1: Synthesis of PA1329

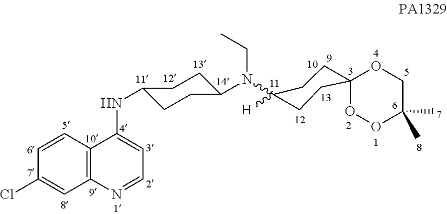

0.2 g (0.4 mmole) of PA1103 was dissolved in 11 ml of CH₂Cl₂ then 73 μl (1.3 mmoles) of acetaldehyde was added under argon at ambient temperature. 0.55 g (2.6 mmoles) of NaBH(OAc)₃ was then added. The reaction medium was stirred at ambient temperature for 2 h. Another 36 μl (0.6 mmoles) of acetaldehyde and 0.27 g (1.3 mmoles) of NaBH(OAc)₃ were added and the mixture was stirred for 2 h. The mixture was purified by silica column chromatography (eluent: ethyl acetate/Et₃N, 80/20, v/v). The phases containing PA1329 were combined and washed with 200 ml of distilled water, dried over Na₂SO₄, filtered and evaporated. The powder obtained was identified as PA1329: 0.13 g (Yield=63%).

Melting point: 165° C. (deg).

¹H NMR (200 MHz, 298K, CDCl₃): δ, ppm: 8.50 (d, J=5.4 Hz, 1H, HC2′), 7.94 (d, J=2.0 Hz, 1H, HC8′), 7.61 (dd, J=9.1 Hz, J=2.7 Hz, 1H, HC5′), 7.38-7.31 (m, 1H, HC6′), 6.42 and 6.41 (d, J=5.5 Hz, 1H, HC3′), 4.78 (d, J=7.6 Hz, 1H, HN), 3.90-3.30 (m, 2H+1H, HC5+HC11′), 2.68-2.54 (m, 1H+2H+2H, HC11+HCcyclohexyl+H₂CN), 2.27 (m, 2H, HCcyclohexyl), 1.86-1.22 (m, 13H+3H, HCcyclohexyl+HC7.8), 1.10 (broad s; 3H, HC7.8), 1.02 and 1.01 (t, J=6.9 Hz, 3H, H₃CH₂N). MS (DCI/NH₃>0): m/z (%): 448.5 (MH⁺, 100%).

### 10—Synthesis of PA 1333, (Figure 10)

### (7-chloro-quinolin-4-yl)-{3-[(3,3-dimethyl-1,2,5-trioxa-spiro[5.5]undec-9-ylamino)-methyl]-adamantane-1-ylmethyl}-amine

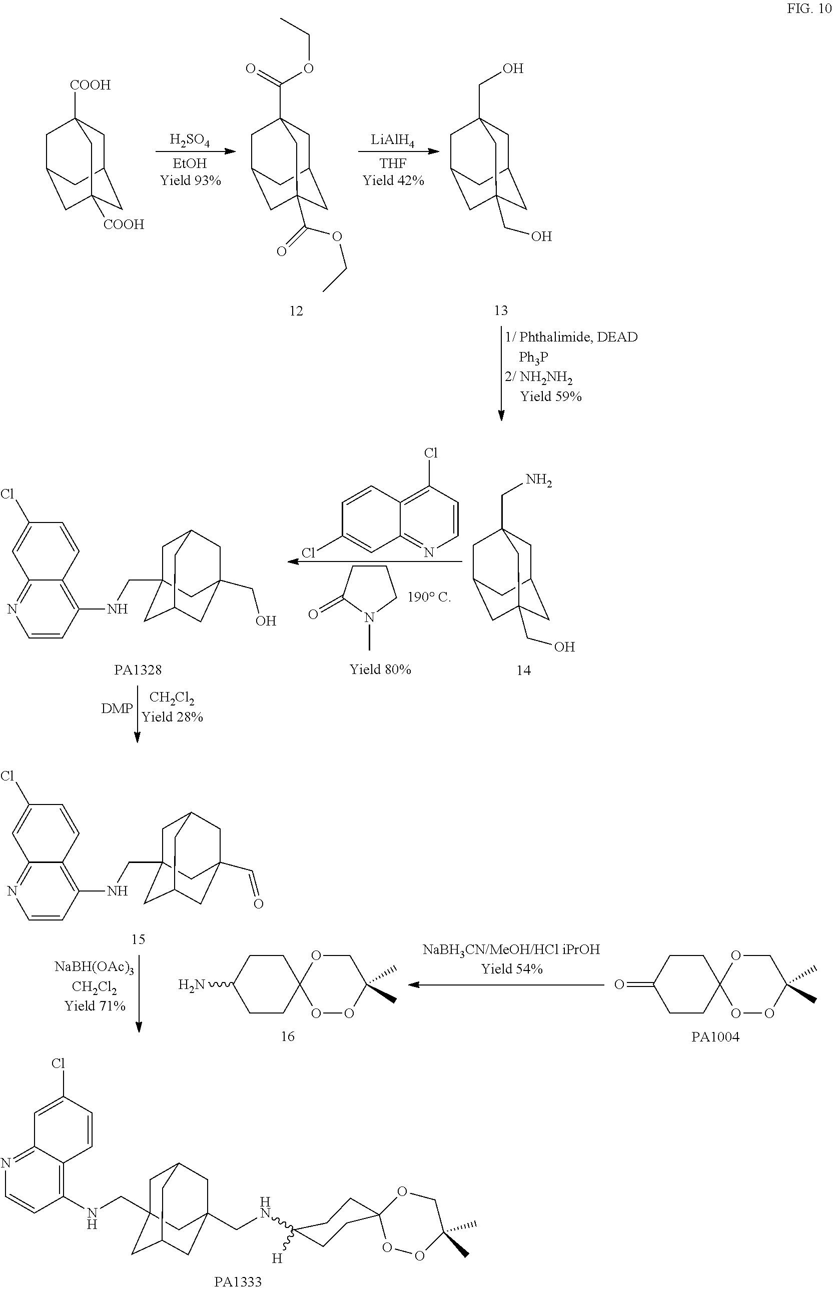

### 10-1: Synthesis of the ethyl di-ester of adamantane-1,3-dicarboxylic acid 12

2.4 g (10 mmoles) of adamantane-1,3-dicarboxylic acid and 4 ml of concentrated sulphuric acid in 100 ml of 95% ethanol were heated under reflux for 9 h. The mixture was allowed to return to ambient temperature. 50 ml of NH₄OH was added to the reaction medium then the solvents were evaporated off. The dry residue was taken up in 100 ml of water saturated with NaCl then extracted with 200 ml of CH₂Cl₂. The organic phase was dried over Na₂SO₄, filtered and concentrated to produce an oil identified as 12: 2.6 g (Yield=93%).

### 10-2: Synthesis of (3-hydroxymethyl-adamantan-1-yl)-methanol 13

2.1 g (7.6 mmoles) of 12 was dissolved in 10 ml of dry THF. This solution was added dropwise, under argon, over one hour, to a solution, cooled in an ice bath, of 25 ml of dry THF and 30 ml (30 mmoles) of 1M LiAlH₄ in ether. When addition was complete, the mixture was refluxed for 1 h30. The reaction medium was hydrolysed and 400 ml of ether was added. The recovered organic phase was dried over MgSO₄, filtered and evaporated to produce a powder identified as 13: 0.63 g (Yield=42%).

### 10-3: Synthesis of (3-hydroxymethyl-adamantan-1-yl)-methanol 14

A solution containing 0.62 g (3.2 mmoles) of 13 and 1.3 ml (6.7 mmoles) of diisopropyl azodicarboxylate was prepared in 50 ml of dry THF under argon. 1.79 g (6.7 mmoles) of PPh₃ and 0.99 g (6.7 mmoles) of phthalimide were added to this solution. The mixture was stirred for 24 h under argon at ambient temperature. The solvents were then evaporated off and the residue was dissolved in 50 ml of methanol. 1.2 ml (13 mmoles) of hydrazine in 35% aqueous solution was added to this solution. The solution was heated under reflux for 15 h. After returning to ambient temperature, the solvents were evaporated off and the white solid obtained was dissolved in 50 ml of an aqueous acetic acid solution at a pH of 4. The suspension obtained was filtered and the pH of the filtrate was brought to a pH of 14 by adding KOH pellets. This aqueous phase was extracted with 200 ml of CH₂Cl₂, the organic phase was dried over Na₂SO₄, filtered and evaporated. Next, the impure product obtained was purified by silica column chromatography (eluent: CH₂Cl₂/MeOH/NH₃aq, 80/20/1, v/v/v). The phases containing 14 were combined, evaporated to produce a solid identified as 14: 0.37 g (Yield=59%).

### 10-4: Synthesis of PA1328

0.3 g (1.6 mmoles) of 4,7-dichloroquinoline and 0.37 g (1.9 mmoles) were heated to 190° C. in 5 ml of N-methylpyrrolidinone for 3 h. The mixture was allowed to return to ambient temperature and 25 ml of water and 0.15 g (3.7 mmoles) of NaOH were added. An oily residue was recovered. This impure product was purified by silica column chromatography (eluent: CH₂Cl₂/Et₃N, 80/20, v/v). The phases containing PA1328 were combined, evaporated and the liquid residue obtained was poured onto 50 ml of water. A precipitate appeared and the filtrate was eliminated. After vacuum drying, said precipitate was re-dissolved in 1 ml of CH₂Cl₂ and 20 ml of n-hexane was added. The precipitate which formed was filtered and vacuum dried to produce a powder identified as PA1328: 0.46 g (Yield=80%).

Melting point: 170° C. (deg).

### 10-5: Synthesis of 3-[(7-chloro-quinolin-4-ylamino)-methyl]-adamantane-1-carboxaldehyde 15

0.45 g (1.3 mmoles) of PA1328 was dissolved in 10 ml of dry CH₂Cl₂. 5.3 ml of a solution of Dess-Martin periodinane (assayed to about 0.5 M) in CH₂Cl₂ was added to that solution, at ambient temperature and under argon. The mixture was stirred for 1 h30 then 5.3 ml of a solution of Dess-Martin periodinane (assayed to about 0.5 M) in CH₂Cl₂ was again added. The mixture was stirred for 30 min then purified directly by silica column chromatography (eluent: ethyl acetate/Et₃N, 90/10, v/v). The phases containing 15 were combined. The organic phase was washed with 200 ml of distilled water then dried over Na₂SO₄, filtered and evaporated to produce a powder identified as 15: 0.13 g (Yield=28%).

### 10-6: Synthesis of 3,3-dimethyl-1,2,5-trioxa-spiro[5.5]undec-9-ylamine 16

5 ml of dry methanol under argon, 0.52 g (2.6 mmoles) of PA1004, 2 g (26 mmoles) of ammonium acetate and 1 ml of 4 Å sieve which had been dried were mixed in a 50 ml flask. 0.16 g (2.6 mmoles) of powdered NaBH₃CN was added. The mixture was stirred for 24 h. 10 ml of distilled water were then added and the pH was brought to a pH of 2 by adding a 6M HCl solution. When gas release had ceased, the pH was raised to 8 by adding a KOH solution. The mixture was extracted with 100 ml of dichloromethane, the organic phase was recovered and dried over Na₂SO₄, filtered then evaporated to produce an oil identified as 16: 0.28 g, (Yield=54%).

### 10-7: Synthesis of PA1333

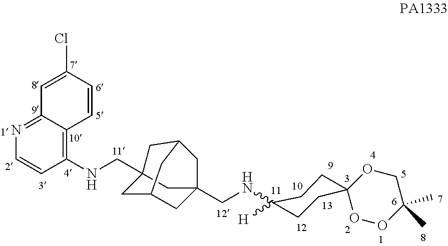

0.13 g (0.36 mmole) of 15 and 0.10 g (0.43 mmole) of 16 were mixed in 5 ml of CH₂Cl₂. The mixture was stirred under argon for 30 min. Next, 0.15 g (0.71 mmoles) of NaBH(OAc)₃ was added and the mixture was stirred for 10 min. 20 μl (0.35 mmole) of acetic acid were then added to the mixture which was stirred at ambient temperature for 12 h. The mixture was purified by silica column chromatography (eluent: ethyl acetate/Et₃N, 98/2, v/v). The phases containing PA1333 were combined. The organic phase was washed with 200 ml of distilled water then dried over Na₂SO₄, filtered and evaporated. 1 ml of an n-hexane/Et₂O, 50/50, v/v mixture was added to the impure product obtained, then the solvents were evaporated off to produce a powder identified as PA1333: 0.14 g (Yield=71%).

Melting point: 95° C.

¹H NMR (200 MHz, 298K, CDCl₃): δ, ppm: 8.47 (d, J=5.4 Hz, 1H, HC2′), 7.92 (d, J=1.0 Hz, 1H, HC8′), 7.83 (d, J=9.2 Hz, 1H, HC5′), 7.34 (dd, J=8.9 Hz, J=2.0 Hz, 1H, HC6′), 6.41 (d, J=5.5 Hz, 1H, HC3′), 5.23 (m, 1H, NH), 3.90-3.30 (m, 2H, HC5), 3.00 (d, J=2.9 Hz, 2H, HC11′), 2.57 (m, 1H, HC11), 2.32-1.16 (m, 2H+14H+8H+3H+1H, HC12′+HCadamantane+HCcyclohexyl+HC7.8+NH), 1.10 (s, 3H, HC7.8). MS (DCI/NH₃>0): m/z (%): 540.5 (MH⁺, 100%).

### 11—Synthesis of PA 1335, (Figure 11)

### (7-chloro-quinolin-4-yl)-N′-(3,3-dimethyl-1,2,5-trioxa-spiro[5.5]undec-9-yl)-octahydro-pentalene-2,5-diamine

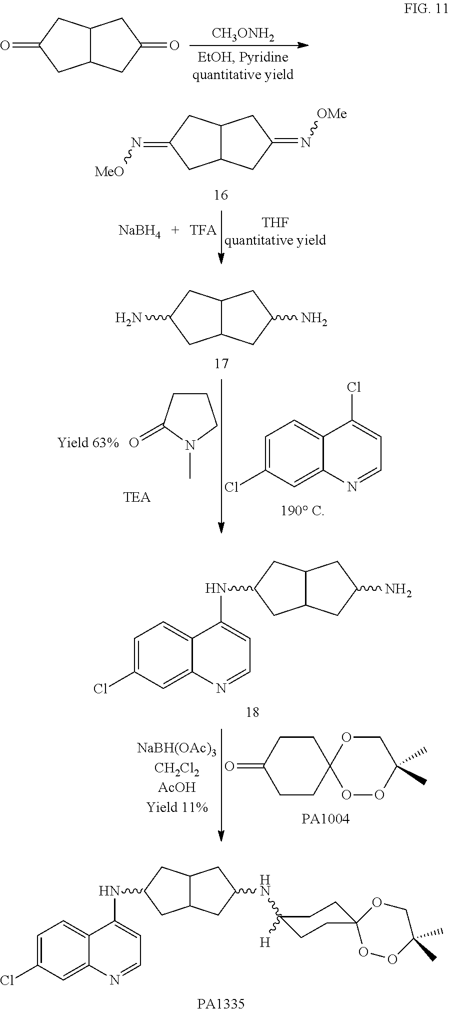

### 11-1: Synthesis of tetrahydro-pentalene-2,5-dione bis-(O-methyl-oxime) 16

2 g (14.4 mmoles) of cis-bicyclo[3.3.0]octane-3,7-dione was dissolved in 21 ml of 95% ethanol then 3.63 g (43.4 mmoles) of methoxylamine hydrochloride and 21 ml of pyridine were added. The mixture was heated under reflux for 18 h. Next, 42 ml of distilled water was added and the mixture was extracted with 126 ml of ether. The organic phase was dried over Na₂SO₄, filtered and the solvents were evaporated off to produce compound 16: 1.96 g (Yield=quantitative).

### 11-2: Synthesis of octahydro-pentalene-2,5-diamine 17

25 ml of THF and 1.93 g (51 mmoles) of powdered NaBH₄ were introduced into a 250 ml flask, under argon at ambient temperature. 3.8 ml (51 mmoles) of trifluoroacetic acid was slowly added. When the effervescence had ceased, a solution of 1 g (5.1 mmoles) of 16 dissolved in 14 ml of dry THF was added dropwise. When addition was complete, the mixture was heated under reflux for 14 h. The reaction medium was then poured onto 65 ml of distilled water and KOH pellets were added to the mixture to produce a pH of 14. The medium was extracted with 260 ml of CH₂Cl₂, the organic phase was dried over Na₂SO₄, filtered and the solvents were evaporated off to produce compound 17: 0.7 g (Yield=quantitative).

### 11-3: Synthesis of N²-(7-chloro-quinolin-4-yl)-octahydro-pentalene-2,5-diamine 18

0.26 g (1.3 mmoles) of 4,7-dichloroquinoline was dissolved in 2 ml of N-methylpyrrolidinone. 0.74 g (5.3 mmoles) of 17 and 741 μl (5.3 mmoles) of triethylamine were added to this solution; the mixture was then heated to 190° C. for 3 h20. The mixture was allowed to return to ambient temperature and 10 ml of 1M NaOH followed by 60 ml of distilled water were added to the reaction medium. This mixture was stirred for 2 h. A pasty residue appeared. This residue was recovered, dissolved in a minimum volume of CH₂Cl₂ and precipitated by adding n-hexane. The precipitate was filtered then vacuum dried to produce a compound identified as 18: 0.25 g (Yield=63%).

### 11-4: Synthesis of PA1335

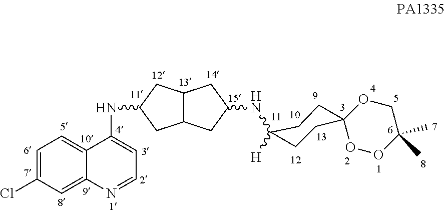

0.23 g (0.76 mmole) of 18 and 0.15 g (0.76 mmole) of PA1004 were mixed in 19 ml of CH₂Cl₂. The mixture was stirred in argon for 24 h. Next, 0.23 g (1.1 mmoles) of NaBH(OAc)₃ and 44 μl (0.76 mmole) of acetic acid were added and the mixture was stirred at ambient temperature for 12 h. Next, 19 ml of a saturated aqueous NaHCO₃ solution was added. The organic phase was recovered, dried over Na₂SO₄, filtered and the solvents were evaporated off. The impure product obtained was purified by silica column flash chromatography (eluent: ethyl acetate/Et₃N, gradient: 5 min: ethyl acetate/Et₃N 98/2, v/v; 5 to 30 min: ethyl acetate/Et₃N 98/2, v/v to ethyl acetate/Et₃N 95/5, v/v; 30 to 40 min: ethyl acetate/Et₃N 95/5, v/v; 40 to 60 min: ethyl acetate/Et₃N 95/5, v/v to ethyl acetate/Et₃N 90/10, v/v; 60 to 70 min: ethyl acetate/Et₃N 90/10, v/v). The phases containing PA1335 were combined, washed with 200 ml of distilled water, dried over Na₂SO₄, filtered and evaporated to produce a powder identified as PA1335: 0.04 g (Yield=11%).

Melting point: 110° C.

¹H NMR (200 MHz, 298K, CDCl₃): δ, ppm: 8.49 (d, J=5.4 Hz, 1H, HC2′), 7.92 (d, J=2.1 Hz, 1H, HC8′), 7.63 (d, J=9.0 Hz, 1H, HC5′), 7.34 (dd, J=9.0 Hz, J=2.2 Hz, 1H, HC6′), 6.43 (d, J=5.4 Hz, 1H, HC3′), 5.12 (d, J=6.7 Hz, 1H, NH), 4.10-3.25 (m, 2H+1H, HC5+HC11′), 2.49 (m, 1H+1H+1H, HC11+HC15′+NH), 2.28-1.11 (m, 10H+8H+6H, HCoctahydro-pentalene+HCcyclohexyl+HC7.8). MS (DCI/NH₃>0): m/z (%): 486 (MH⁺, 100%).

In addition to compounds the production protocols for which have been detailed in the foregoing, other compounds with formula (I) in accordance with the invention are shown in Table 2 below; these examples are not limiting and serve only to illustrate the invention.

TABLE 1

| | | Melting | |
|---|---|---|---|
| | | point | ¹H NMR |
| N° | Compound | (° C.) | (δ, ppm) |
| 1 | 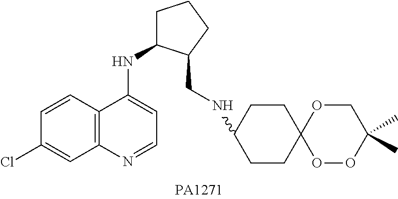 | 102 | N-(7-chloro-quinolin-4-yl)-cis-2-[(3,3-dimethyl-1,2,5- trioxa-spiro[5.5]undec-9-ylamino)-methyl]- cyclopentylamine 250 MHz, 298K, CDCl₃: 9.31 (m, 1H), 8.39 (m, 1H), 7.97 (s, 1H), 7.87-7.80 (m, 1H), 7.45-7.36 (m, 1H), 6.29 (d, J = 5.7 Hz, 1H), 3.99 (m, 2H), 3.80-3.43 (m, 2H), 3.10-1.12 (m, 21H), 1.11 (s, 3H) |
| 2 | 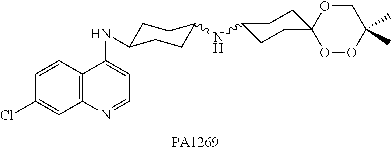 | 130 | N-(7-chloro-quinolin-4-yl)-N′-(3,3-dimethyl-1,2,5-trioxa- spiro[5.5]undec-9-yl)-cyclohexane-1,4-diamine 250 MHz, 298K, CDCl₃: 8.55 and 8.51 (d, J = 5.4 Hz, 1H), 7.97-7.94 (m, 1H), 7.69 and 7.62 (d, J = 7.7 Hz, 1H), 7.39-7.32 (m, 1H), 6.48 and 6.42 (d, J = 5.5 Hz, 1H), 5.06 and 4.83 (d, J = 6.0 Hz, 1H), 3.90-3.43 (m, 2H + 1H), 2.87-2.21 (m, 1H + 1H + 1H), 2.25-1.18 (m, 15H + 3H + 1H), 1.03 (broad s, 3H) |
| 3 | 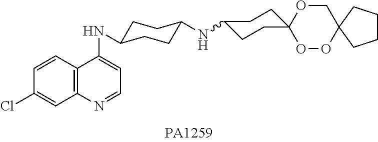 | 191 | N-(7-chloro-quinolin-4-yl)-N′-(6,7,14-trioxa- dispiro[4.2.5.2]pentadec-11-yl)-cyclohexane-trans-1,4- diamine 250 MHz, 298K, CDCl₃: 8.52 (d, J = 5.4 Hz, 1H), 7.95 (d, J = 2.0 Hz, 1H), 7.63 (d, J = 9.0 Hz, 1H), 7.35 (dd, J = 8.9 Hz, J = 2.1 Hz, 1H), 6.4 (d, J = 5.5 Hz, 1H), 4.8 (m, 1H), 4.10-3.40 (m, 3H), 2.80-1.25 (m, 27H) |
| 4 | 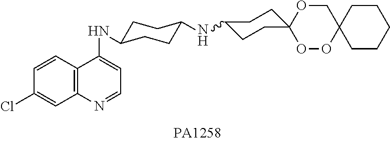 | 197 | N-(7-chloro-quinolin-4-yl)-N′-(7,8,15-trioxa- dispiro[5.2.5.2]hexadec-12-yl)-cyclohexane-trans-1,4- diamine 250 MHz, 298K, CDCl₃: 8.48 (d, J = 5.5 Hz, 1H), 7.95 (s, 1H), 7.74 (m, 1H), 7.35 (d, J = 7.3 Hz, 1H), 6.44 (d, J = 5.5 Hz, 1H), 5.2 (m, 1H), 3.80- 3.40 (m, 3H), 3.05-1.15 (m, 29H) |
| 5 | 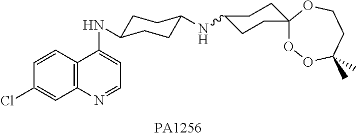 | 171 | N-(7-chloro-quinolin-4-yl)-N′-(9,9-dimethyl-7,8,12-trioxa- spiro[5.6]dodec-3-yl)-cyclohexane-trans-1,4-diamine 250 MHz, 298K, CDCl₃: 8.48 (d, J = 5.4 Hz, 1H), 7.93 (d, J = 2.2 Hz, 1H), 7.64 (d, J = 9.2 Hz, 1H), 7.33 (dd, J = 9.1 Hz, J = 2.2 Hz, 1H), 6.42 (d, J = 5.5 Hz, 1H), 4.92 (d, J = 5.5 Hz, 1H), 3.97-3.82 (m, 1H), 3.72-3.62 (m, 1H), 3.47 (m, 1H), 2.74-1.20 (m, 24H), 1.12 and 1.13 (s, 3H) |
| 6 | 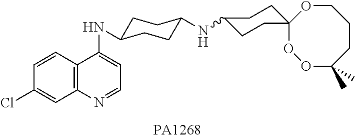 | 167 | N-(7-chloro-quinolin-4-yl)-N′-(9,9-dimethyl-7,8,13-trioxa- spiro[5.7]tridec-3-yl)-cyclohexane-trans-1,4-diamine 250 MHz, 298K, CDCl₃: 8.49 (d, J = 5.4 Hz, 1H), 7.94 (d, J = 1.9 Hz, 1H), 7.67 (d, J = 8.7 Hz, 1H), 7.34 (dd, J = 8.9 Hz, J = 2.0 Hz, 1H), 6.43 (d, J = 5.5 Hz, 1H), 5.00 (broad s, 1H), 3.90-3.83 (m, 1H), 3.65- 3.49 (m, 1H + 1H), 2.77 (s, 2H), 2.42 (m, 2H), 2.27-1.30 (m, 19H), 1.30 and 1.29 (s, 3H), 1.11 and 1.08 (s, 3H) |
| 7 | 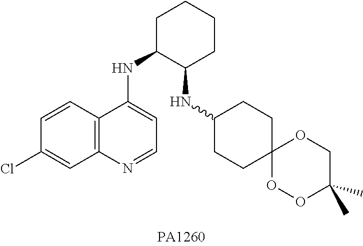 | 85 | N-(7-chloro-quinolin-4-yl)-N′-(3,3-dimethyl-1,2,5-trioxa- spiro[5.5]undec-9-yl)-cyclohexane-cis-1,2-diamine 250 MHz, 298K, CDCl₃: 8.45 (d, J = 5.5 Hz, 1H), 7.93 (s, 1H), 7.69 and 7.66 (d, J = 9.2 Hz, 1H), 7.39 and 7.35 (dd, J = 8.9 Hz, J = 2.1 Hz, 1H), 6.41 (d, J = 5.8 Hz, 1H), 6.35 and 6.34 (d, J = 5.2 Hz, 1H), 3.90-3.30 (m, 4H), 3.03-1.20 (m, 21H), 1.13 (s, 3H) |
| 8 | 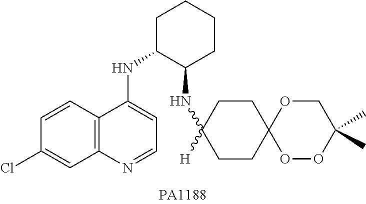 | 129 | N-(7-chloro-quinolin-4-yl)-N′-(3,3-dimethyl-1,2,5-trioxa- spiro[5.5]undec-9-yl)-cyclohexane-trans-1,2-diamine 250 MHz, 298K, CDCl₃: 8.46 (d, J = 5.5 Hz, 1H), 7.92 (d, J = 1.8 Hz, 1H), 7.76 and 7.72 (d, J = 9.6 Hz, 1H), 7.38 and 7.35 (dd, J = 7.1 Hz, J = 2.1 Hz, 1H), 6.47 and 6.46 (d, J = 5.5 Hz, 1H), 5.80 (m, 1H), 3.90-3.40 (m, 2H), 3.18 (m, 1H), 2.77 (m, 2H), 2.40-1.17 (m, 23H) |
| 9 | 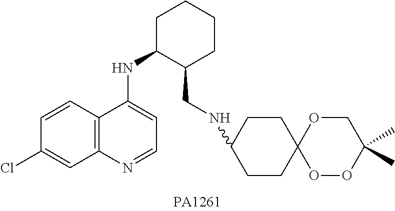 | 132 | N-(7-chloro-quinolin-4-yl)-cis-2-[(3,3-dimethyl-1,2,5- trioxa-spiro[5.5]undec-9-ylamino)-methyl]- cyclohexylamine 250 MHz, 298K, CDCl₃: 9.50 (m, 1H), 8.30 and 8.28 (d, J = 6.0 Hz, 1H), 8.12 and 8.09 (s, 1H), 7.85 and 7.83 (d, J = 8.5 Hz, 1H), 7.45 and 7.31 (d, J = 9.6 Hz, 1H), 6.31 and 6.29 (d, J = 6.1 Hz, 1H), 3.79 (m, 2H), 3.65-3.43 (m, 2H), 3.31 (t, J = 11.7 Hz, 1H), 2.83 (d, J = 12 Hz, 2H), 2.64 (m, 2H), 2.23 (m, 2H), 2.00- 1.17 (m, 16H), 1.11 (s, 3H) |
| 10 | 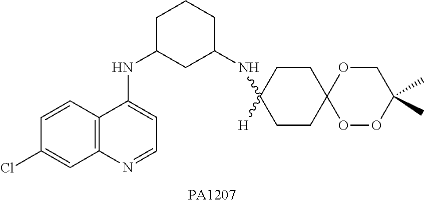 | 177 | N-(7-chloro-quinolin-4-yl)-N′-(3,3-dimethyl-1,2,6-trioxa- spiro[5.5]undec-9-yl)-cyclohexane-1,3-diamine 250 MHz, 298K, CDCl₃: 8.52 and 8.47 (d, J = 5.4 Hz, 1H), 7.93 (s, 1H), 7.70 and 7.62 (d, J = 8.8 Hz, 1H), 7.36-7.31 (m, 1H), 7.04 and 4.89 (m and d, J = 7.2 Hz, 1H), 6.45 and 6.34 (d, J = 5.4 Hz, 1H), 4.24-3.43 (m, 3H), 3.08 (m, 1H), 2.89-2.63 (m, 2H), 2.08- 1.13 (m, 22H) |
| 11 | 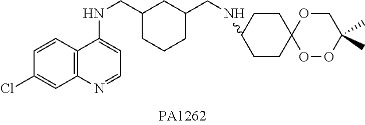 | 150 | (7-chloro-quinolin-4-yl)-{3-[(3,3-dimethyl-1,2,5-trioxa- spiro[5.5]undec-9-ylamino)-methyl]-cyclohexylmethyl}- amine 250 MHz, 298K, CDCl₃: 8.52 (d, J = 5.3 Hz, 1H), 7.94 (d, J = 2.1 Hz 1H), 7.67 (d, J = 8.9 Hz, 1H), 7.36 (dd, J = 1.7 Hz, J = 8.9 Hz, 1H), 6.39 (d, J = 5.3 Hz, 1H), 5.07 (s, 1H), 3.90-3.30 (m, 3H), 3.16 (t, J = 5.9 Hz, 1H), 2.90-2.40 (m, 4H), 1.90-1.10 (m, 24H) |
| 12 | 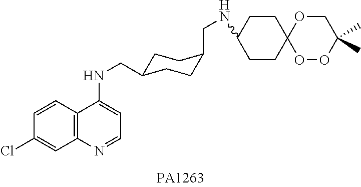 | 118 | (7-chloro-quinolin-4-yl)-{4-[(3,3-dimethyl-1,2,5-trioxa- spiro[5.5]undec-9-ylamino)-methyl]-cyclohexylmethyl}- amine 250 MHz, 298K, CDCl₃: 8.52 (d, J = 5.3 Hz, 1H), 7.96 (d, J = 2.2 Hz 1H), 7.67 (d, J = 9.0 Hz, 1H), 7.37 (dd, J = 2.2 Hz, J = 8.9 Hz, 1H), 6.42 (d, J = 5.3 Hz, 1H), 5.04 (s, 1H), 3.90-3.30 (m, 3H), 3.25 (t, J = 6.7 Hz, 2H), 2.59 (d, J = 6.7 Hz, 2H), 1.94-1.18 (m, 22H), 1.16 (s, 3H) |
| 13 | 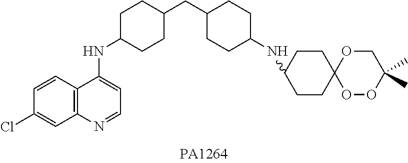 | 220 | (7-chloro-quinolin-4-yl)-{4-[4-(3,3-dimethyl-1,2,5-trioxa- spiro[5.5]undec-9-ylamino)-cyclohexylmethyl]- cyclohexyl}-amine 250 MHz, 298K, CDCl₃: 8.51-8.48 (m, 1H), 7.95-7.93 (m, 1H), 7.67-7.60 (m, 1H), 7.38-7.31 (m, 1H), 6.43-6.40 (m, 1H), 5.05 and 4.83 (d, J = 6.7 Hz, 1H), 3.79-3.38 (m, 3H), 2.80-2.40 (m, 3H), 2.22-0.88 (m, 34H) |
| 14 | 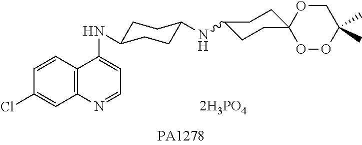 | 210 | N-(7-chloro-quinolin-4-yl)-N′-(3,3-dimethyl-1,2,5-trioxa- spiro[5.5]undec-9-yl)-cyclohexane-trans-1,4-diamine, diphosphate 400 MHz, 300K, DMSOd₆: 8.41 and 8.39 (d, 1H), 8.37 and 8.34 (d, 1H), 7.78 (d, 1H), 7.46-7.43 (m, 1H), 7.00-6.98 (m, 1H) 6.58 and 6.57 (d, 1H) 3.90-3.40 (m, 2H + 1H), 3.14 (m, 1H), 3 .01 (m, 1H), 2.20-1.00 (m, 22H) |
| 15 | 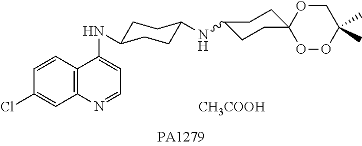 | 187 | N-(7-chloro-quinolin-4-yl)-N′-(3,3-dimethyl-1,2,5-trioxa- spiro[5.5]undec-9-yl)-cyclohexane-trans-1,4-diamine, diacetate 400 MHz, 300K, DMSOd₆: 8.36 (d, 1H), 8.35 and 8.33 (d, 1H), 7.76 (d, 1H), 7.43 and 7.41 (dd, 1H), 6.92 and 6.90 (m, 1H), 6.51 (d, 1H), 3.80-3.40 (m, 2H + 1H), 2.7 (m, 1H), 2.55 (m, 1H), 2.10-1.89 (m, 4H), 1.88 (s, 6H of AcOH), 1.76-1.69 (m, 2H), 1.47-1.00 (m, 16H) |
| 16 | 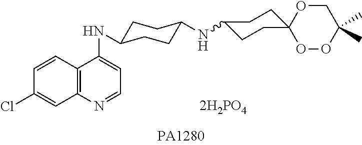 | 166 | N-(7-chloro-quinolin-4-yl)-N′-(3,3-dimethyl-1,2,5-trioxa- spiro[5.5]undec-9-yl)-cyclohexane-trans-1,4-diamine, disulphate 400 MHz, 300K, DMSOd₆ 8.65 and 8.62 (d, 1H), 8.55 and 8.54 (d, 1H), 8.36 (s, 1H), 7.93 and 7.92 (d, 1H), 7.78-7.76 (m, 1H), 7.01 and 7.00 (d, 1H), 3.81-3.20 (m, 5H), 2.53-1.03 (m, 22H) |
| 17 | 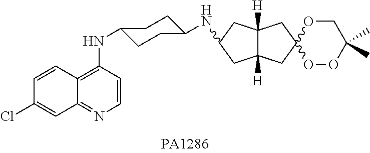 | 148 | N-(7-Chloro-quinolin-4-yl)-N′-[-5-spiro-(3,3-dimethyl- 1,2,5-trioxan-6-yl)-octahydro-cis-pentalen-2-yl]- cyclohexane-trans-1,4-diamine 250 MHz, 298K, CDCl₃: 8.50 (d, J = 5.4 Hz, 1H), 7.94 (d, J = 2.1 Hz, 1H), 7.62 (d, J = 8.9 Hz, 1H), 7.35 (d, J = 1.9 Hz, J = 8.8 Hz, 1H), 6.43 (d, J = 5.5 Hz, 1H), 4.82 (d, J = 7.0 Hz, 1H), 3.70-3.40 (m, 3H), 3.11 (m, 1H), 2.65-2.50 (m, 2H), 2.24-1.25 (m, 21H), 0.87 (s, 3H) |
| 18 | 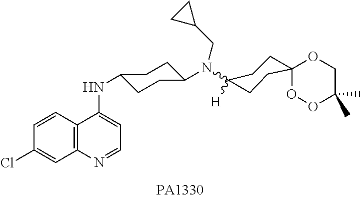 | 176 | N-(7-chloro-quinolin-4-yl)-N′-cyclopropylmethyl-N′-(3,3- dimethyl-1,2,5-trioxa-spiro[5.5]undec-9-yl)-cyclohexane- 1,4-diamine 200 MHz, 298K, CDCl₃: 8.49 (d, J = 5.4 Hz, 1H), 7.93 (d, J = 2.1 Hz, 1H), 7.61 and 7.59 (d, J = 8.9 Hz, 1H), 7.37-7.31 (m, 1H), 6.41 and 6.40 (d, J = 5.5 Hz, 1H), 4.77 (d, J = 7.1 Hz, 1H), 4.90-3.30 (m, 3H), 2.78 (m, 2H), 2.44 (d, J = 6.1 Hz, 2H), 2.26-2.21 (m, 2H), 1.80-1.20 (m, 17H), 1.09 (s, 3H), 0.76 (m, 1H), 0.52- 0.41 (m, 2H), 0.11-0.03 (m, 2H) |
| 19 | 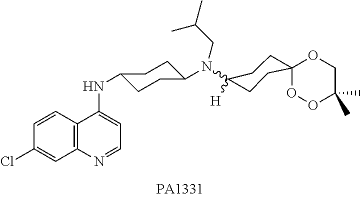 | 157 | N-(7-chloro-quinolin-4-yl)-N′-(3,3-dimethyl-1,2,5-trioxa- spiro[5.5]undec-9-yl)-N′-isobutyl-cyclohexane-1,4- diamine 200 MHz, 298K, CDCl₃: 8.49 (d, J = 5.4 Hz, 1H), 7.93 (d, J = 2.2 Hz, 1H), 7.61 and 7.59 (d, J = 9.0 Hz, 1H), 7.37-7.31 (m, 1H), 6.42 and 6.39 (d, J = 5.5 Hz, 1H), 4.77 (d, J = 7.6 Hz, 1H), 4.90-3.30 (m, 3H), 2.59 (m, 2H), 2.26 (d, J = 7.3 Hz, 2H), 1.75-0.81 (m, 29H) |
| 20 | 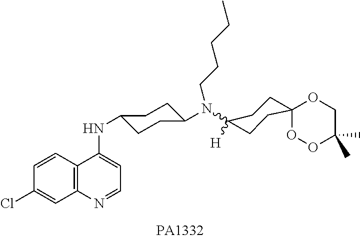 | 135 | N-(7-chloro-quinolin-4-yl)-N′-(3,3-dimethyl-1,2,5-trioxa- spiro[5.5]undec-9-yl)-N′-pentyl-cyclohexane-1,4-diamine 200 MHz, 298K, CDCl₃: 8.49 (d, J = 5.3 Hz, 1H), 7.93 (d, J = 2.1 Hz, 1H), 7.62 and 7.61 (d, J = 9.0 Hz, 1H), 7.37-7.32 (m, 1H), 6.42 and 6.41 (d, J = 5.4 Hz, 1H), 4.80 (d, J = 7.2 Hz, 1H), 4.90-3.30 (m, 3H), 2.65 (m, 2H), 2.48 (t, J = 6.7 Hz, 2H), 2.25 (m, 2H), 1.79 (m, 3H), 1.59-1.10 (m, 23H), 0.88 (t, J = 5.0 Hz, 3H) |
| 21 | 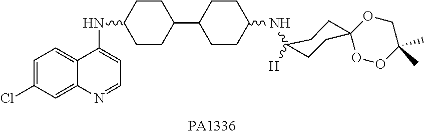 | 177 | N⁴-(7-chloro-quinolin-4-yl)-N⁴-[(3,3-dimethyl-1,2,5- trioxa-spiro[5.5]undec-9-yl)-bicyclohexyl-4,4′-diamine 200 MHz, 298K, CDCl₃: 8.49 (d, J = 5.4 Hz, 1H), 7.93 (d, J = 2.0 Hz, 1H), 7.65 (d, J = 9.0 Hz, 1H), 7.35 (dd, J = 8.8 Hz and J = 2.0 Hz, 1H), 6.42 (d, J = 5.4 Hz, 1H), 4.48 (m, 1H), 3.90-3.20 (m, 3H), 2.86- 2.50 (m, 3H), 2.20-1.04 (m, 32H) |
| 22 | 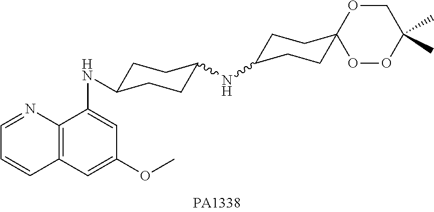 | 55 | N-(3,3-dimethyl-1,2,5-trioxa-spiro[5.5]undec-9-yl)-N′-6- methoxy-quinolin-8-yl)-cyclohexane-1,4-diamine 200 MHz, 298K, CDCl₃: 8.53-8.49 (m, 1H), 7.91 (td, J = 8.3 Hz, J = 2.0 Hz, 1H), 7.32-7.27 (m, 1H), 6.33-6.26 and 6.02 (m, 2H + 1H), 3.87 (s, 3H), 3.66-3.31 (m, 3H), 2.72 (m, 2H), 2.29 (m, 2H), 2.03- 1.16 (m, 21H) |

A study of the pharmacological properties of the coupling products with formula (I) of the invention has shown that they exhibit antimalarial activity.

Obtaining such an effect is all the more advantageous since resistance to strains of Plasmodium falciparum, the deadly species, with respect to antimalarial medicinal products is developing and further, vaccine protection, on which a huge amount of research is being been carried out, may not be delivered for several years.

A. Study of Antimalarial Activity of Dual Molecules of the Invention on P. falciparum

We report below the in vitro results obtained on P. falciparum cultivated in human red blood corpuscles.

1. Culture of P. falciparum

Strains of P. falciparum were cultivated continuously using Trager and Jensen's method (Science, 1976, 193, 673-675): the parasites were maintained in human red blood corpuscles (O±), diluted to a blood parasite level of 2% in RPMI 1640 medium supplemented with 25 mM Hepes+24 mM NaHCO₃+2 mM L-glutamine and complemented with 5% human serum from all groups.

The parasites were incubated at 37° C., in a moist atmosphere and 5% CO₂. FcB1-Columbia and FcM29-Cameroon strains are, respectively, moderately (IC₅₀: 66 nM) and very strongly (IC₅₀: 258 nM) chloroquine-resistant. The IC₅₀ for artemisinin on these two strains are, respectively, 11 nM and 5 nM.

2. Chemosensitivity Test

The antimalarial activity tests were carried out using the radioactive micromethod of Desjardins et al. (Antimicrob. Agents Chemother., 1979, 16, 710-718). Each molecule was tested in triplicate. The tests were carried out in 96 well microplates. The P. falciparum strains were cultured in RPMI 1640 solutions complemented with 5% human serum with haematocrit at 2% and a blood parasite level of 1.5%. For each test, the parasites were incubated with decreasing concentrations of test compounds for 48 h at 37° C., in a moist atmosphere and 5% CO₂. The artemisinin and chloroquine diphosphate were used as reference molecules. The first dilution of the test compounds was carried out at 1 mg/ml in dimethylsulphoxide. The dilution series for the successive daughter solutions was also prepared in dimethylsulphoxide. Each daughter dilution was then diluted to 1/50^{th} in RPMI 1640 complemented with 5% human serum, all of the dilutions being made at 37° C. Said dilutions were then added to the parasites in culture in the microplates. After adding the test compound, the parasites were cultured in RPMI 1640 with 5% human serum and 1% dimethylsulphoxide. Parasite growth was measured by the incorporation of tritiated hypoxanthine (added 24 h after beginning exposure to the test compound) and compared with the incorporation in the absence of the test compound (taken as 100%). The values for IC₅₀ (concentrations required to inhibit parasite growth by 50%) were determined by plotting the percentage inhibition as a function of the logarithm of the dose using GraphPad Prism 4® processing software (GraphPad software, Inc., 5755 Oberlin Drive, #110, San Diego, Calif. 92121, USA).

3. Results

The IC₅₀ for compounds with formula (I) of the invention were below 1 μM. For the strains employed, for the majority of test compounds with formula (I), this IC₅₀ was comparable with that of artemisinin or even better.

No notable difference was measured between IC₅₀s for the test compounds on one or other of the strains, namely on the FcB1-Colombia strain (strain moderately resistant to chloroquine) and on the FcM29-Cameroon strain (strain highly resistant to chloroquine).

As an example, the IC₅₀ values for compounds of example 1 on the FcM29-Cameroon strain were, respectively, equal to 6 nM for PA1103 and 4 nM for PA1188.

The invention envisages exploiting the properties of the compounds of the invention for use as a medicinal product and for the production of pharmaceutical compositions with antimalarial properties.

B. Metabolic Stability Study

The compounds of the invention were tested as regards their metabolic stability on human hepatic microsomes, by comparison with prior art compounds.

These experiments were carried out on human hepatic microsomes in the presence of NADPH cofactor, necessary for the activity of the principal enzymes, namely cytochromes P-450 (CYP) and flavine mono-oxygenases (FMO). In the presence of NADPH, the test substrates underwent oxidative biotransformation reactions. After 20 minutes, the reaction was stopped by adding 1 volume of acetonitrile.

The supernatant was then removed after centrifuging (speed 3000 g for 10 minutes at 4° C.).

The supernatant was analysed by high performance liquid chromatography coupled to a mass spectrometer (LC-MS/MS) and the degradation of each of the test compounds was calculated as a percentage (%) with respect to T₀.

1. Preparation of Human Hepatic Microsomal Fractions

The microsomal fractions were prepared from human hepatic tissue deriving from at least 12 different donors and frozen at −80° C.

The tissue was defrosted then dried, weighed and cut into thin sheets before homogenizing.

The tissue was homogenized using a Potter-Elvejheim type homogenizer at +4° C. The tissue homogenates were then centrifuged at 10000 g for 30 minutes at +4° C. The supernatant was centrifuged at 105 000 g for 1 hour at +4° C. The residue was finally taken up into suspension in a final volume of KH₂PO₄/K₂HPO₄ buffer containing 20% (v/v) of glycerol (1 ml for 2 grams of tissue). The hepatic microsomal fractions obtained were divided into aliquots (500 μl), frozen rapidly in liquid nitrogen and kept frozen at −80° C. until use.

2. Incubation of Microsomes

Incubation Conditions:

concentration of microsomal proteins: 1 mg/ml;

concentration of BSA (bovine serum albumin (BSA): 1 mg/ml;

concentration of substrate (test compound): 5 μM;

CYP and FMO co-factors: 1 mM NADPH;

Phosphate buffer (pH 7.4): 10 mM.
The reaction was initiated by adding 1 mM of NADPH and incubating for 20 minutes at 37° C., with stirring. The reaction was stopped by adding 1 volume of cold acetonitrile.
Total incubation volume=300 μl

3. Results

The results are shown in Table 2 below:

TABLE 2

| | | Percentage |
|---|---|---|
| Compound | Structure | metabolization |
| Invention: Example 1 | 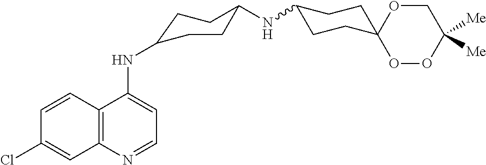 | 11% |
| WO01/77105: Example 6 (DU1302) | 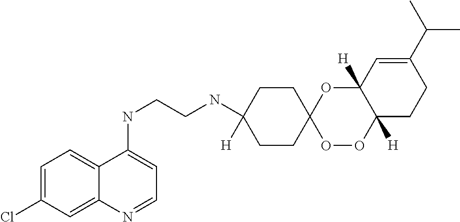 | 95-100% |
| WO2005/049619: PA1110 | 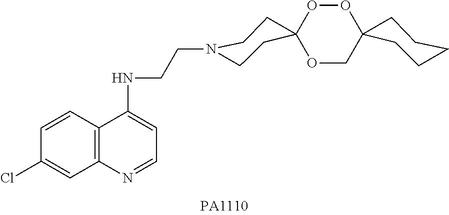 | 98% |
| artesunate | 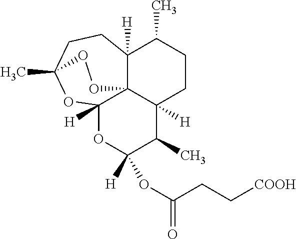 | 100% |
| chloroquine | 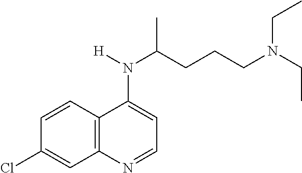 | 29% |

According to the results shown in Table 2, the compound of Example 1 of the invention is about 3 times less degraded than chloroquine and about 10 times less degraded than the prior art compounds.

The compound of Example 1 of the invention is much more stable in human hepatic microsomes than the other test compounds.

Thus, the compounds of the invention, in addition to their good antimalarial activity advantageously have very good metabolic stability, rendering the compounds of the invention particularly advantageous for therapeutic use.

Thus, in another aspect, the invention pertains to medicinal products which comprise a compound with formula (I), or an addition salt thereof with a pharmaceutically acceptable acid, or a hydrate or a solvate of the compound with formula (I).

Said medicinal products have therapeutic use, in the prevention of and treatment of malaria.

In a further aspect, the present invention concerns pharmaceutical compositions comprising a compound of the invention as an active principle. Said pharmaceutical compositions contain an effective dose of at least one compound with formula (I) of the invention, or a pharmaceutically acceptable salt, a hydrate or solvate of said compound, and at least one pharmaceutically acceptable excipient. Said excipients are selected, as a function of the pharmaceutical form and the desired mode of administration, from the usual excipients which are known to the skilled person.

In the pharmaceutical compositions of the present invention for oral, sublingual, subcutaneous, intramuscular, intravenous, topical, local, intratracheal, intranasal, transdermal or rectal administration, the active principle with formula (I) above, or its optional salt, solvate or any hydrate, may be administered in a unitary administration form, mixed with conventional pharmaceutical excipients, to prevent or treat malaria.

Suitable unitary administration forms include oral forms such as tablets, soft or hard gelules, powders, granules and oral solutions or suspensions, sublingual, buccal, intratracheal, intraocular or intranasal, administration forms, forms for inhalation, topical, transdermal, subcutaneous, intramuscular or intravenous forms, rectal forms of administration and implants. For topical application, it is possible to use the compounds of the invention in creams, gels, ointments or lotions. Preferably, administration is carried out orally, rectally or by injection.

As an example, one unitary form of administering a compound of the invention in the form of a tablet may comprise the following components:

| | |
|---|---|
| Compound of the invention | 50.0 mg |
| Mannitol | 223.75 mg |
| Sodium croscaramellose | 6.0 mg |
| Corn starch | 15.0 mg |
| Hydroxypropyl methyl cellulose | 2.25 mg |
| Magnesium stearate | 3.0 mg |

There may be particular cases where higher or lower doses are appropriate; such doses fall within the scope of the invention. As is usual, the dose which is appropriate for each patient is determined by the physician as a function of the mode of administration and the weight and response of that patient.

In a further aspect, the present invention also concerns a method for treating or preventing malaria which comprises administering to a patient an effective dose of a compound with formula (I) in accordance with the invention, or one of its pharmaceutically acceptable salts, hydrates or solvates.

The invention also pertains to biological reagents the active principles of which are constituted by the compounds of the invention. These reagents may be used as references or standards in any antimalarial activity studies.

What is claimed is:

1. A compound according to formula (II: 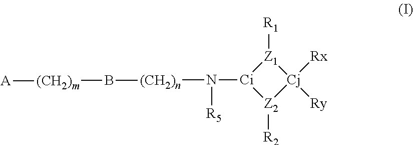
wherein:
A represents:
(a) a residue of a molecule with antimalarial activity comprising an aminoquinoline according to formula (IIa): 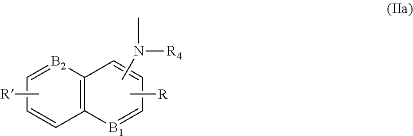
in which:
R and R′, which may be identical or different, each represents one or more substituents, occupying distinct positions on the cycles to which they are attached, selected from:
(i) a hydrogen or halogen atom, a —OH, —CF₃, —OCF₃, aryl, —O-aryl, heteroaryl, alkyl or —O-alkyl group, said alkyl groups containing 1 to 5 carbon atoms; or
(ii) a cycloalkyl or —O-cycloalkyl group, said cycloalkyl groups possibly containing 3 to 5 carbon atoms; or
(iii)-NO₂ or —N(Rₐ,R_{b}), wherein:
Rₐ, and R_{b}, which may be identical or different, each independently represents a hydrogen atom or an alkyl group containing 1 to 5 carbon atoms; or
Rₐ, and R_{b}, which may be identical or different, each represents a cycloalkyl group which may contain 3 to 5 carbon atoms; or
Rₐ and R_{b} together with the nitrogen atom to which they are attached form a pyrrolidinyl or piperidinyl group;
R₄ represents a hydrogen atom or an alkyl group which may contain 1 to 5 carbon atoms or R₄ represents a cycloalkyl group which may contain 3 to 5 carbon atoms, and
B₁ represents a nitrogen atom and B₂ represents a —CH=link, or B₁ represents a —CH=link and B₂ represents a nitrogen atom;
(b) a residue of a molecule with antimalarial activity comprising a group according to formula (IIIa):
R₆—CHOH— (IIIa)
in which:
R₆ represents an aryl radical, preferably a 9-phenanthrenyl or a nitrogenous heterocyclic residue, preferably a 4-quinolinyl optionally substituted with one or more groups R as defined for residue according to formula (IIa); or
(c) a residue facilitating bioavailability, said residue having one or more heteroatoms selected from N, O and S in a mono- or poly-cyclic molecule which may contain 6 to 18 carbon atoms, which may be saturated or unsaturated or in a chain which may contain 1 to 18 linear carbon atoms, optionally substituted, such as a guanidinium, morpholino, peptide or polyamine residue;
B represents:
(a) a cycloalkyl group which may contain 3 to 8 carbon atoms, optionally substituted with one or more groups selected from: a halogen atom, a hydroxyl group, an alkyl group which may contain 1 to 6 carbon atoms, or a cycloalkyl group which may contain 3 to 6 carbon atoms; or
(b) a bi- or tri-cyclic group which may contain 4 to 18 carbon atoms, optionally substituted with one or more groups selected from a halogen atom, a hydroxyl group, an alkyl group which may contain 1 to 6 carbon atoms, or a cycloalkyl group which may contain 3 to 6 carbon atoms; or
(c) 2 cycloalkyl groups which may contain 3 to 6 carbon atoms, said cycloalkyl groups being connected together via a single bond or an alkylene chain which may contain 1 or 2 carbon atoms;
m and n each independently represents 0, 1 or 2;
R₅ represents:
(a) a hydrogen atom; or
(b) an alkyl group, a —C(O)-alkyl group or a —C(O)O-alkyl group, said alkyl groups possibly containing 1 to 5 carbon atoms; or
(c) a cycloalkyl group, a —C(O)-cycloalkyl group, a —C(O)β-cycloalkyl group or a C₁₋₃-alkylene-cycloalkyl group, said cycloalkyl groups possibly containing 3 to 6 carbon atoms;
Z₁ and Z₂, which may be identical or different, each represents an alkylene radical which may contain 1 to 4 saturated or unsaturated carbon atoms, the entity Z₁+Z₂+Ci+Cj thus representing either:
a cycloalkyl group which may contain 3 to 10 carbon atoms; or
a poly-cyclic structure which may contain 4 to 18 carbon atoms;
or Z₁ or Z₂ optionally represent a single bond between the carbon atoms Ci and Cj, provided that that Z₁ and Z₂ cannot both represent a single bond at the same time;
R₁ and R₂, which may be identical or different, each represents a hydrogen atom or a functional group which is capable of enhancing hydrosolubility;
Rₓ and R_{y} together form a cyclic peroxide containing 4 to 8 links and containing 1 or 2 supplemental oxygen atoms in the cyclic structure, Cj being one of the vertices of said cyclic peroxide;
wherein said cyclic peroxide is substituted with a group R₃, R₃ representing 1 to 8 groups which may be identical or different, occupying any positions on the carbon atoms of the peroxide cycle and being selected from:
(i) hydrogen, halogen, a —OH, —CF₃, —NO₂, —OCF₃, aryl, —O-aryl, heteroaryl, alkyl or —O-alkyl group, said alkyl groups containing 1 to 10 carbon atoms;
(ii) a cycloalkyl group possibly containing 3 to 7 carbon atoms and possibly further containing 1 to 3 heteroatoms selected from oxygen, nitrogen and sulphur, optionally substituted with one or more groups selected from a halogen atom, a hydroxyl group, an alkyl group which may contain 1 to 8 carbon atoms or a cycloalkyl group which may contain 3 to 8 carbon atoms;
(iii) an —O-cycloalkyl group which may contain 3 to 7 carbon atoms; or
(iv) a bi- or tri-cyclic group which may contain 4 to 18 carbon atoms and may also contain 1 to 6 heteroatoms selected from oxygen, nitrogen and sulphur, optionally substituted with one or more groups selected from a halogen atom, a hydroxyl group, an alkyl group which may contain 1 to 8 carbon atoms or a cycloalkyl group which may contain 3 to 8 carbon atoms;
or two R₃ groups occupying adjacent carbon atoms on the peroxide cycle may together form a saturated or unsaturated cycloalkyl group containing 5 or 6 carbon atoms, said group R₃ itself possibly being substituted with 1 to 6 substituents R₃ as defined above;
or two R₃ groups occupying the same carbon atom of the peroxide cycle may together form a cycloalkyl group which may contain 3 to 7 carbon atoms or a bi- or tri-cyclic group which may contain 4 to 18 carbon atoms;
as a base or addition salt with an acid, as a hydrate or solvate, in the racemic, isomeric or mixed form, as well as diastereoisomers and mixtures thereof.

2. A compound according to claim 1, wherein R₅ represents:
a hydrogen atom;
an alkyl group, a —C(O)-alkyl group or a —C(O)O-alkyl group, said alkyl groups possibly containing 1 to 5 carbon atoms; or
a cycloalkyl group, a —C(O)-cycloalkyl group or a —C(O)β-cycloalkyl group, said cycloalkyl groups possibly containing 3 to 6 carbon atoms;
as a base or addition salt with an acid, as a hydrate or solvate, in the racemic, isomeric and mixed form, as well as diastereoisomers and mixtures thereof.

3. A compound according to claim 1 or claim 2, wherein:
A represents an aminoquinoline according to formula (IIa): 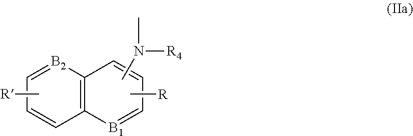
wherein:
R and R′, which may be identical or different, each represents one or more substituents occupying distinct positions on the cycles to which they are attached, said substituents selected from:
(i) a hydrogen or halogen atom, an —OH, —CF₃, —OCF₃, aryl, —O-aryl, heteroaryl, alkyl or —O-alkyl group, said alkyl groups containing 1 to 5 carbon atoms;
(ii) a cycloalkyl group or —O-cycloalkyl group, said cycloalkyl groups possibly containing 3 to 5 carbon atoms; and
(iii) —NO₂ or —N(Rₐ,R_{b}), wherein:
Rₐ and R_{b}, which may be identical or different, represent hydrogen atoms or an alkyl group containing 1 to 5 carbon atoms; or
Rₐ, and R_{b}, which may be identical or different, each represents a cycloalkyl group which may contain 3 to 5 carbon atoms; or
Rₐ, and R_{b} together with the nitrogen atom to which they are attached form a pyrrolidinyl or piperidinyl group;
R₄ represents a hydrogen atom, an alkyl group which may contain 1 to 5 carbon atoms, or a cycloalkyl group which may contain 3 to 5 carbon atoms; and
B₁ represents a nitrogen atom and B₂ represents a —CH=link, or B₁ represents a —CH=link and B₂ represents a nitrogen atom;
as a base or addition salt with an acid, as a hydrate or solvate, in the racemic, isomeric and mixed form, as well as diastereoisomers and mixtures thereof.

4. A compound according to claim 3, wherein A represents an aminoquinoline according to formulae (IIb) or (IIc): 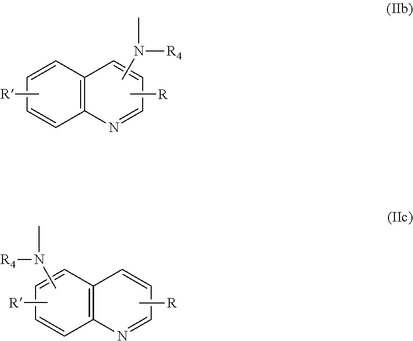
as a base or addition salt with an acid, as a hydrate or solvate, in the racemic, isomeric and mixed form, as well as diastereoisomers and mixtures thereof.

5. A compound according to claim 1 or claim 2, wherein B represents cis-1,2-methylenecyclopentyl, trans-1,2-cyclohexyl, cis-1,2-cyclohexyl, cis-1,2-methylenecyclohexyl, trans-1,4-cyclohexyl, cis-1,4-cyclohexyl, a cis/trans-1,4-cyclohexyl mixture, a cis/trans-1,3-cyclohexyl mixture, a cis/trans-1,3-dimethylenecyclohexyl mixture, cis-1,4-dimethylenecyclohexyl, or 4,4′-methylene-bis-cyclohexane; as a base or addition salt with an acid, as a hydrate or solvate, in the racemic, isomeric and mixed form, as well as diastereoisomers and mixtures thereof.

6. A compound according to claim 1 or claim 2 and having formula (I.1): 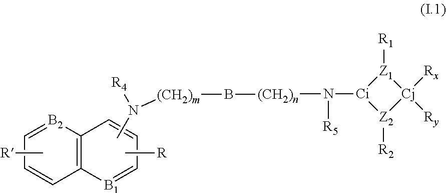
as a base or addition salt with an acid, as a hydrate or solvate, in the racemic, isomeric and mixed form, as well as diastereoisomers and mixtures thereof.

7. A compound according to claim 6, wherein B represents cis-1,2-methylenecyclopentyl, trans-1,2-cyclohexyl, cis-1,2-cyclohexyl, cis-1,2-methylenecyclohexyl, trans-1,4-cyclohexyl, cis-1,4-cyclohexyl, a cis/trans-1,4-cyclohexyl mixture, a cis/trans-1,3-cyclohexyl mixture, a cis/trans-1,3-dimethylenecyclohexyl mixture, cis-1,4-dimethylenecyclohexyl, or 4,4′-methylene-bis-cyclohexane; as a base or addition salt with an acid, as a hydrate or solvate, in the racemic, isomeric and mixed form, as well as diastereoisomers and mixtures thereof.

8. A compound according to claim 1 or claim 2 and having formula (I.2): 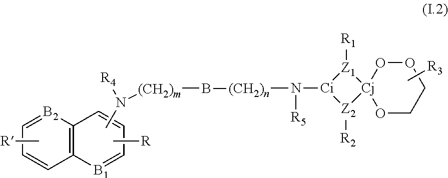
as a base or addition salt with an acid, as a hydrate or solvate, in the racemic, isomeric and mixed form, as well as diastereoisomers and mixtures thereof.

9. A compound according to claim 8, wherein B represents cis-1,2-methylenecyclopentyl, trans-1,2-cyclohexyl, cis-1,2-cyclohexyl, cis-1,2-methylenecyclohexyl, trans-1,4-cyclohexyl, cis-1,4-cyclohexyl, a cis/trans-1,4-cyclohexyl mixture, a cis/trans-1,3-cyclohexyl mixture, a cis/trans-1,3-dimethylenecyclohexyl mixture, cis-1,4-dimethylenecyclohexyl, or 4,4′-methylene-bis-cyclohexane; as a base or addition salt with an acid, as a hydrate or solvate, in the racemic, isomeric and mixed form, as well as diastereoisomers and mixtures thereof.

10. A compound according to claim 1 or claim 2 and having formula (I.3): 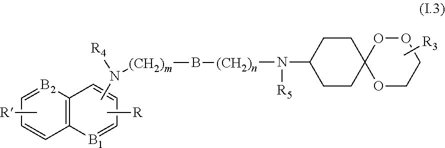
as a base or addition salt with an acid, as a hydrate or solvate, in the racemic, isomeric and mixed form, as well as diastereoisomers and mixtures thereof.

11. A compound according to claim 10, wherein B represents cis-1,2-methylenecyclopentyl, trans-1,2-cyclohexyl, cis-1,2-cyclohexyl, cis-1,2-methylenecyclohexyl, trans-1,4-cyclohexyl, cis-1,4-cyclohexyl, a cis/trans-1,4-cyclohexyl mixture, a cis/trans-1,3-cyclohexyl mixture, a cis/trans-1,3-dimethylenecyclohexyl mixture, cis-1,4-dimethylenecyclohexyl, or 4,4′-methylene-bis-cyclohexane; as a base or addition salt with an acid, as a hydrate or solvate, in the racemic, isomeric and mixed form, as well as diastereoisomers and mixtures thereof.

12. A compound according to claim 1 or claim 2, wherein:
A represents an aminoquinoline with formulae (IIb) or (IIc): 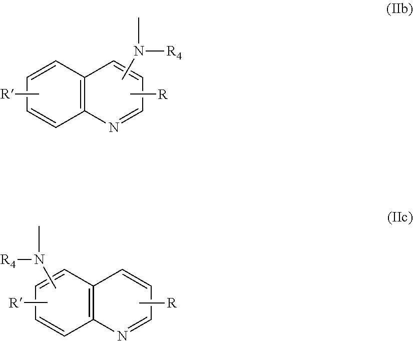
B represents:
(i) a cycloalkyl group which may contain 3 to 8 carbon atoms, optionally substituted with one or more groups selected from: a halogen atom, a hydroxyl group, an alkyl group which may contain 1 to 6 carbon atoms or a cycloalkyl group which may contain 3 to 6 carbon atoms; or
(ii) 2 cycloalkyl groups which may contain 3 to 6 carbon atoms, said cycloalkyls being connected together via a single bond or an alkylene chain which may contain 1 or 2 carbon atoms;
m and n each independently represents 0, 1 or 2;
R₅ represents a hydrogen atom;
R₁ and R₂ each represents a hydrogen atom;
Rₓ and R_{y} together form a cyclic peroxide comprising 4 to 8 links and comprising 1 or 2 supplemental oxygen atoms in the cyclic structure, Cj being one of the vertices of said cyclic peroxide;
wherein said cyclic peroxide is substituted with a group R₃, R₃ representing 1 to 8 identical or different groups, occupying any positions on the carbon atoms of the peroxide cycle and being selected from hydrogen, halogen, an —OH, —CF₃, —NO₂, —OCF₃, aryl, —O-aryl, heteroaryl, alkyl and —O-alkyl group, said alkyl groups containing 1 to 10 carbon atoms,
or two groups R₃ occupying by the same carbon atom of the peroxide cycle may together form a cycloalkyl group which may contain 3 to 7 carbon atoms or a bi- or tri-cyclic group which may contain 4 to 18 carbon atoms;
as a base or addition salt with an acid, as a hydrate or solvate, in the racemic, isomeric and mixed form, as well as diastereoisomers and mixtures thereof.

13. A compound according to claim 1, wherein said compound is:
PA1103, PA1265, PA1251, PA1252, PA1253, PA1255, PA1271, PA1269, PA1259, PA1258, PA1256, PA1268, PA1260, PA1188, PA1261, PA1207, PA1262, PA1263, or PA1264.

14. A compound according to claim 1, wherein said compound is:
PA1305, PA1308, PA1329, PA1333, PA1335, PA1278, PA1279, PA1280, PA1286, PA1330, PA1331, PA1332, PA1336.

15. A process for preparing compounds according to formula (I), said process comprising the step of reacting a compound having formula (III): 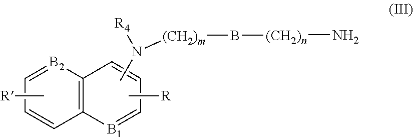
in which B, R, R′, B₁, B₂ and R₄ are as defined for the compound with formula (IIa) and B, m and n are as defined for the compound with formula (I); with a compound having formula (II): 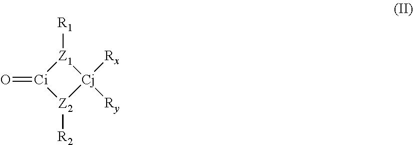
in which R₁, R₂, Z₁, Z₂, Rₓ and R_{y} are as defined in compounds with formula (I).

16. A compound according to formula (III): 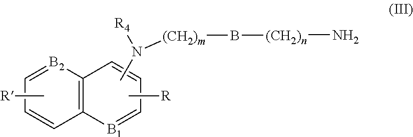
in which B, R, R′, B₁, B₂ and R₄ are as defined for the compound with formula (IIa) and B, m and n are as defined for the compound with formula (I).

17. A pharmaceutical composition comprising a compound according to any one of claim 1, 2, 4, 7, 9, 11, 13 or 14, or a pharmaceutically acceptable salt, hydrate or solvate thereof, and at least one pharmaceutically acceptable excipient.

18. A pharmaceutical composition comprising a compound according to claim 3, or a pharmaceutically acceptable salt, hydrate or solvate thereof, and at least one pharmaceutically acceptable excipient.

19. A pharmaceutical composition comprising a compound according to claim 5, or a pharmaceutically acceptable salt, hydrate or solvate thereof, and at least one pharmaceutically acceptable excipient.

20. A pharmaceutical composition comprising a compound according to claim 6, or a pharmaceutically acceptable salt, hydrate or solvate thereof, and at least one pharmaceutically acceptable excipient.

21. A pharmaceutical composition comprising a compound according to claim 8, or a pharmaceutically acceptable salt, hydrate or solvate thereof, and at least one pharmaceutically acceptable excipient.

22. A pharmaceutical composition comprising a compound according to claim 10, or a pharmaceutically acceptable salt, hydrate or solvate thereof, and at least one pharmaceutically acceptable excipient.

23. A pharmaceutical composition comprising a compound according to claim 12, or a pharmaceutically acceptable salt, hydrate or solvate thereof, and at least one pharmaceutically acceptable excipient.

24. A method for treating or preventing malaria in a subject, said method comprising administering to said subject a therapeutically effective amount of a compound according to any one of claim 1, 2, 4, 7, 9, 11, 13 or 14, or of a pharmaceutically acceptable salt, hydrate or solvate thereof.

25. A method for treating or preventing malaria in a subject, said method comprising administering to said subject a therapeutically effective amount of a compound according to claim 3, or of a pharmaceutically acceptable salt, hydrate or solvate thereof.

26. A method for treating or preventing malaria in a subject, said method comprising administering to said subject a therapeutically effective amount of a compound according to claim 5, or of a pharmaceutically acceptable salt, hydrate or solvate thereof.

27. A method for treating or preventing malaria in a subject, said method comprising administering to said subject a therapeutically effective amount of a compound according to claim 6, or of a pharmaceutically acceptable salt, hydrate or solvate thereof.

28. A method for treating or preventing malaria in a subject, said method comprising administering to said subject a therapeutically effective amount of a compound according to claim 8, or of a pharmaceutically acceptable salt, hydrate or solvate thereof.

29. A method for treating or preventing malaria in a subject, said method comprising administering to said subject a therapeutically effective amount of a compound according to claim 10, or of a pharmaceutically acceptable salt, hydrate or solvate thereof.

30. A method for treating or preventing malaria in a subject, said method comprising administering to said subject a therapeutically effective amount of a compound according to claim 12, or of a pharmaceutically acceptable salt, hydrate or solvate thereof.
